(19) United States
(12) Patent Application Publication
(10) Pub. No.: US 2019/0030226 A1
(43) Pub. Date: Jan. 31, 2019

(54) AUGMENTED PRESSURE THERAPY FOR WOUNDS

(71) Applicant: EDWARD D. LIN, OSPREY, FL (US)

(72) Inventor: EDWARD D. LIN, OSPREY, FL (US)

(21) Appl. No.: 15/663,709

(22) Filed: Jul. 29, 2017

(51) Int. Cl. A61M 1/00; A61F 13/02; A61M 35/00; A61L 15/44; A61L 15/58; A61L 15/24; A61L 15/26

(52) U.S. Cl. CPC A61M 1/0088; A61F 13/0216; A61M 1/0058; A61M 35/00; A61L 15/44; A61M 2202/0208; A61L 15/24; A61L 15/26; A61M 2205/3331; A61M 2205/3337; A61M 2205/50; A61L 15/58

(57) ABSTRACT

An apparatus for wound therapy is disclosed herein. In various aspects, the apparatus for wound therapy may include a wound interface sealingly engaged with the skin to define an enclosed space surrounding a wound bed at a skin surface of the skin. The enclosed space may be fluid-tight, and the enclosed space may be evacuated to a pressure pₘᵢₙ less than ambient pressure p_{amb} and a condition of essentially no fluid passing through the enclosed space through a port formed about the wound interface in fluid communication through the wound interface with the enclosed space. In various aspects, the apparatus may include fluid input into the enclosed space via the port to increase the pressure p₀ within the enclosed space from the minimum pressure pₘᵢₙ to a maximum pressure pₘₐₓ, the fluid being either a liquid or a gas having an O₂ concentration greater than atmospheric air. The pressure p₀ within the enclosed space may be varied periodically in a pressure cycle between the minimum pressure pₘᵢₙ and the maximum pressure pₘₐₓ where pₘᵢₙ≤p₀≤pₘₐₓ and where pₘᵢₙ≤p_{amb}≤pₘₐₓ by consecutive withdrawal of fluid from the enclosed space and input of fluid into the enclosed space through the port. This Abstract is presented to meet requirements of 37 C.F.R. § 1.72(b) only. This Abstract is not intended to identify key elements of the methods of use and related apparatus disclosed herein or to delineate the scope thereof.

### CROSS-REFERENCE TO RELATED APPLICATIONS

This application hereby incorporates by reference in the entirety herein the co-pending U.S. patent application Ser. No. ______ entitled “DEFORMATION RESISTANT WOUND THERAPY APPARATUS AND RELATED METHODS OF USE,” co-pending U.S. patent application Ser. No. ______ entitled “CONTROL APPARATUS AND RELATED METHODS FOR WOUND THERAPY DELIVERY,” co-pending U.S. patent application Ser. No. ______ entitled “WOUND COVER APPARATUS AND RELATED METHODS OF USE,” and co-pending U.S. patent application Ser. No. ______ entitled “WOUND THERAPY APPARATUS WITH SCAR MODULATION PROPERTIES AND RELATED METHODS,” all by Edward D. Lin as inventor and applicant and filed on the same date as the present application.

### BACKGROUND OF THE INVENTION

### Field

The present disclosure relates to medical devices, and, more particularly, to apparatus and related methods for delivering therapy to wound beds.

### Related Art

A wound bed, as used herein, includes a localized region of tissue that has lost skin and been affected by hostile factors, resulting in, for example, cellular abnormalities such as swelling, inflammation, degradation, infection, or cell death. The wound bed may include varying degrees of exposure of underlying layers and structures, along with possible infections and tissue changes. The wound bed represents an unhealed wound. In contrast, a healed wound is a skin surface that was previously injured but the focal breach is now entirely sealed and covered by varying amounts of epidermis and scar tissue. The wound bed may lie within a wound boundary that extends around the affected region on the skin surface of the skin. The wound bed may extend contiguously in depth within the dermis, and the wound bed may extend subcutaneously, for example, into fat, muscle, or beyond. Thus, the wound bed may include undermined flaps, sinuses, tunnels, and fistulae and the surrounding affected tissues. An example of a wound bed including some reference anatomy is illustrated in FIG. 1. Wound boundary, as used herein, refers to the boundary of the wound bed at a skin surface of the skin.

Various negative pressure wound therapy (NPWT) devices are currently used for treatment of wound bed that includes a dressing, a sheet, and an evacuation tube. In order to use current NPWT devices, the wound bed is packed with the dressing and the evacuation tube is placed about the dressing. The sheet is then placed over the wound bed and attached adhesively to the skin surface around the wound bed to seal the wound bed, dressing, and evacuation tube in place. Finally, air within the region between the sheet and the wound bed is evacuated through the evacuation tub, which is in fluid communication with the dressing, to produce a suction pressure pₛ within an enclosed space between the sheet and the wound bed that is less than the ambient pressure p_{amb}. The wound bed and surrounding skin are compressed as the suction pressure pₛ. is decreased below the ambient pressure p_{amb}. Exudate from the wound bed may be transmitted through the dressing and then evacuated through the evacuation tube. The wound bed may be subjected to a suction pressure pₛ that is static and typically between around −80 mm Hg to around −175 mm Hg below ambient pressure p_{amb}.

The suction pressure pₛ may be maintained statically continually for weeks, if not months, until end of therapy, except during dressing changes. Because capillaries are exceedingly thin-walled microscopic tubules, capillaries are easily collapsed shut by the suction pressure pₛ. Studies have shown that the blood flow is actually diminished by 30-50%, in direct proportion to suction pressure pₛ in tissue at 0.5 cm distance from the wound bed but increased by up to 40% in wound tissue between 1 cm and 2.5 cm from the wound bed.

It has thus become recognized that it may be beneficial to relieve the suction pressure pₛ from time to time in order to allow capillaries adjacent to the wound bed to refill. However, the relief of the suction pressure pₛ, if provided at all, is accomplished in current NPWT devices by input of atmospheric air into the enclosed space between the sheet and the wound bed. The suction pressure pₛ may be relieved, for example, to p_{amb}−25 mm Hg instead of to p_{amb} in order to maintain the sheet in sealing securement over the wound bed. Such relief of the suction pressure pₛ in some devices may occur only intermittently, or not at all.

NPWT in conjunction with instillation has been used as a way of reducing bioburden and dead tissue in the wound bed. In NPWT with instillation, an individually premeasured aliquot of irrigation liquid corresponding to the wound bed volume is, for example, infused into the wound dressing through the evacuation tube. The liquid is allowed to remain for between 10-20 minutes, and then NPWT is resumed, which withdraws the irrigation liquid from the dressing. This instillation may provide a benefit akin to a “micro-debridement” without necessitating a costly trip to the operating room. Following a case review, a panel of national wound care experts made consensus recommendations including: that instillation volume be limited to 10-100 cc, or until foam dressing is visibly soaked, and that static suction, not intermittent suction be used at −125 to −150 mm Hg. One authority recommended the use of unusually high −300 to −600 mm Hg suction pressure pₛ. In all instances, no NWPT is given during the instillation therapy. Nevertheless, instillation in conjunction with NWPT is shown to reduce the number of visits to the operating room for debridement from 3 to 2.6 and the number of days since last surgical debridement from 9.8 to 7.5 days.

NWPT on average lasts almost 6 months, attesting to the challenges of getting enough blood flow and oxygen to the wound bed to enable healing. NWPT requires skilled nursing and physician supervision, and NWPT may require delivery within a hospital or other such institutional setting. NWPT frequently fails resulting in tens of thousands of deaths due to wound-related complications and 80,000 limb amputations per year in the US, each of which may represent many months, if not years, of failed costly therapy. NPWT may be difficult to apply, and dressing changes are often exceedingly painful because of the disruption to granulation tissue that occurs with each dressing change that may typically occur every other day. Such disruption to the granulation tissue may retard the healing process. About 66% of wound beds require 15 weeks of NWPT while another 10% require 33 weeks or more of NWPT to heal.

In addition, the evacuation tube may become clogged by the proteinaceous exudate, which may result in interruption of the NWPT. The suction pressure pₛ may be inaccurately sensed indicating that suction pressure pₛ is at the desired amount when in fact there is little or no suction pressure within the enclosed space over the wound bed. Because the dressing is tedious to apply and painful to remove, as a practical matter, it is deemed not feasible to remove the dressing repeatedly in order to attach other devices that deliver other therapies

Another type of wound therapy in common use is hyperbaric oxygen (HBO). The patient is placed in a total body hyperbaric chamber and exposed, typically, to 2.5 ATA (atmospheres absolute) of medically pure oxygen for 90 minutes. Exposure past 120 minutes increases the risk of oxygen toxicity, probably due to the increased formation of superoxide, H₂O₂, or other oxidizing free radicals. Seizures and other serious consequences may result. Such a 90-minute session provides oxygen enrichment to the wound bed for a mere 6% of a day. The Medicare branch of the US Government usually approves HBO treatment for 40 sessions at a time at a cost per session exceeding $1,000. This emphasizes not only the high cost of chronic wound care and HBO's low ability to effect healing with just a few sessions, but also the general lack of a more efficacious therapeutic modalities.

Therefore, for at least these reasons, it is evident that there is a strong and unmet need for improved apparatus for wound therapy as well as related methods of wound therapy and related compositions of matter.

### BRIEF SUMMARY OF THE INVENTION

These and other needs and disadvantages may be overcome by the wound therapy methods and related apparatus and compositions of matter disclosed herein. Additional improvements and advantages may be recognized by those of ordinary skill in the art upon study of the present disclosure.

An apparatus for wound therapy is disclosed herein. In various aspects, the apparatus for wound therapy may include a wound interface sealingly engaged with the skin to define an enclosed space surrounding a wound bed at a skin surface of the skin. The enclosed space may be fluid-tight, and the enclosed space may be evacuated to a pressure pₘᵢₙ, less than ambient pressure p_{amb} and a condition of essentially no fluid passing through the enclosed space through a port formed about the wound interface in fluid communication through the wound interface with the enclosed space. In various aspects, the apparatus may include fluid input into the enclosed space via the port to increase the pressure p₀ within the enclosed space from the minimum pressure pₘᵢₙ to a maximum pressure pₘₐₓ, the fluid being either a liquid or a gas having an O₂ concentration greater than atmospheric air.

The pressure p₀ within the enclosed space may be varied periodically in a pressure cycle between the minimum pressure pₘᵢₙ and the maximum pressure pₘₐₓ where pₘᵢₙ≤p₀≤pₘₐₓ and where pₘᵢₙ≤p_{amb}≤pₘₐₓ by consecutive withdrawal of fluid from the enclosed space and input of fluid into the enclosed space through the port. The fluid input into the enclosed space to increase the pressure p₀ within the enclosed space from the minimum pressure pₘᵢₙ to the maximum pressure pₘₐₓ has an O₂ concentration greater than atmospheric air, in various aspects. A therapy regimen comprising at least a sequence of pressure cycles of the pressure p₀ within the enclosed space may be delivered to the wound bed. Related methods of use may include the step of providing therapy to the wound bed by delivering one or more pressure cycles to the wound bed within the enclosed space, the one or more pressure cycles may be grouped into a therapy regimen.

This summary is presented to provide a basic understanding of some aspects of the methods and apparatus disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1 by cross-sectional view an exemplary wound bed that demonstrates undermining, wound tunneling, and fistulae;

FIG. 2A illustrates by cut-away perspective view an exemplary implementation of a wound therapy apparatus as may be employed in the various exemplary methods of wound therapy disclosed herein at an exemplary first stage of operation;

FIG. 2B illustrates by cut-away view portions of the exemplary wound therapy apparatus of FIG. 2A;

FIG. 2C illustrates by cut-away view portions of the exemplary wound therapy apparatus of FIG. 2A at an exemplary second stage of operation;

FIG. 3A illustrates by cut-away elevation view another exemplary implementation of a wound therapy apparatus as may be employed in the various exemplary methods of wound therapy disclosed herein at an exemplary first stage of operation;

FIG. 3B illustrates by cut-away elevation view portions of the exemplary wound therapy apparatus of FIG. 3A at an exemplary second stage of operation;

FIG. 4 illustrates by cut-away perspective view a third exemplary implementation of a wound therapy apparatus as may be employed in the various exemplary methods of wound therapy disclosed herein;

FIG. 5 illustrates by cut-away elevation view a fourth exemplary implementation of a wound therapy apparatus as may be employed in the various exemplary methods of wound therapy disclosed herein;

FIG. 6 illustrates a fifth exemplary implementation of a wound therapy apparatus as may be employed in the various exemplary methods of wound therapy disclosed herein;

FIG. 7A illustrates by Cartesian plot an exemplary pressure cycle as may be employed in the various exemplary wound therapy apparatus and methods of wound therapy disclosed herein;

FIG. 7B illustrates by Cartesian plot a second exemplary pressure cycle as may be employed in the various exemplary wound therapy apparatus and methods of wound therapy disclosed herein;

FIG. 7C illustrates by Cartesian plot a third exemplary pressure cycle as may be employed in the various exemplary wound therapy apparatus and methods of wound therapy disclosed herein;

FIG. 7D illustrates by Cartesian plot a fourth exemplary pressure cycle as may be employed in the various exemplary wound therapy apparatus and methods of wound therapy disclosed herein;

FIG. 7E illustrates by Cartesian plot a fifth exemplary pressure cycle as may be employed in the various exemplary wound therapy apparatus and methods of wound therapy disclosed herein;

FIG. 7F illustrates by Cartesian plot a sixth exemplary pressure cycle as may be employed in the various exemplary wound therapy apparatus and methods of wound therapy disclosed herein;

FIG. 7G illustrates by Cartesian plot a seventh exemplary pressure cycle as may be employed in the various exemplary wound therapy apparatus and methods of wound therapy disclosed herein;

FIG. 7H illustrates by Cartesian plot an eighth exemplary pressure cycle as may be employed in the various exemplary wound therapy apparatus and methods of wound therapy disclosed herein;

FIG. 7I illustrates by Cartesian plot a ninth exemplary pressure cycle as may be employed in the various exemplary wound therapy apparatus and methods of wound therapy disclosed herein;

FIG. 7J illustrates by Cartesian plot a tenth exemplary pressure cycle as may be employed in the various exemplary wound therapy apparatus and methods of wound therapy disclosed herein;

FIG. 8 illustrates by process flow chart an exemplary method of use of various exemplary implementations of the wound therapy apparatus, and

FIG. 9 illustrates by process flow chart another exemplary method of use of various exemplary implementations of the wound therapy apparatus.

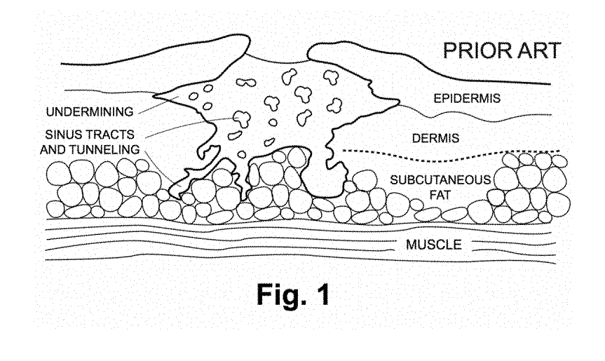

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. Use herein of relative terms such as generally, about, approximately, essentially, may be indicative of engineering, manufacturing, or scientific tolerances such as ±0.1%, ±1%, ±2.5%, ±5%, or other such tolerances, as would be readily recognized by those of ordinary skill in the art upon study of this disclosure.

### DETAILED DESCRIPTION OF THE INVENTION

A wound therapy apparatus and related methods of use of the wound therapy apparatus are disclosed herein. In various aspects, the wound therapy apparatus includes a wound interface sealingly engaged with the skin to define an enclosed space surrounding a wound bed at a skin surface of the skin. The enclosed space may be fluid-tight, and the enclosed space may be evacuated to a pressure pₘᵢₙ less than ambient pressure p_{amb} and at a condition of essentially no fluid passing through the enclosed space. The wound interface includes a port formed about the wound interface that defines a lumen for fluid communication through the wound interface with the enclosed space, in various aspects. The wound therapy apparatus further includes fluid input into the enclosed space of the wound interface via the port to increase the pressure p₀ within the enclosed space from the minimum pressure pₘᵢₙ to a maximum pressure pₘₐₓ, the fluid being either a liquid or a gas having an O₂ concentration greater than atmospheric air, in various aspects.

In various aspects, fluid may be input into the enclosed space through the lumen or fluid may be withdrawn from the enclosed space through the lumen in sequence to vary pressure p₀ within the enclosed space in a pressure cycle between a minimum pressure pₘᵢₙ and a maximum pressure pₘₐₓ where pₘᵢₙ≤p₀≤pₘₐₓ, in various aspects. The fluid input into the enclosed space to increase the pressure p₀ within the enclosed space from the minimum pressure pₘᵢₙ toward the maximum pressure pₘₐₓ has an O₂ concentration greater than atmospheric air, which is generally taken as about 20.95% per volume for dry air or about 0.2095 mole oxygen per mole of air, in various aspects. In various aspects, pₘᵢₙ≤p_{amb} where pressure p_{amb} in the ambient pressure proximate the wound therapy apparatus. The maximum pressure pₘₐₓ may be greater than the ambient pressure p_{amb}, the maximum pressure pₘₐₓ may be generally equal to the ambient pressure p_{amb}, or maximum pressure pₘₐₓ may be less than ambient pressure p_{amb}, in various aspects. Sequential input of fluid into the enclosed space and withdrawal of fluid from the enclosed space means that input of fluid into the enclosed space and the withdrawal of fluid from the enclosed space does not occur simultaneously. In certain aspects, fluid may be being input into the enclosed space or fluid may be being withdrawn from the enclosed space but not the input of fluid simultaneously with withdrawal of fluid. In certain other aspects, such as during irrigation, liquid may be input into the enclosed space simultaneously with withdrawal of liquid from the enclosed space.

In the apparatus and related methods of use disclosed herein, the pressure p₀ within the enclosed space may be increased from the minimum pressure pₘᵢₙ by input of fluid with an O₂ concentration greater than atmospheric air. Thus, in various aspects, when multiple pressure cycles are applied to the wound bed, the wound bed is exposed to fluid with O₂ concentration greater than atmospheric air during portions of the first pressure cycle as well as during at least portions of the second and subsequent pressure cycles, which may increase the oxygen supply to the wound bed during therapy with resulting therapeutic benefits. In various aspects, the fluid with O₂ concentration greater than atmospheric air may be medical grade oxygen. Medical grade oxygen may conform to certain standards, for example, United States Food and Drug Administration standards or other appropriate regulatory standards. In various aspects, the medical grade oxygen may be United States Pharmacopoeia grade oxygen.

In other aspects, the fluid input into the enclosed space to increase the pressure p₀ within the enclosed space from the minimum pressure pₘᵢₙ to the maximum pressure pₘₐₓ may be a liquid with therapeutic benefit.

Relief of the pressure, for example from the minimum pressure pₘᵢₙ to the maximum pressure pₘₐₓ, by either fluid with O₂ concentration greater than atmospheric air or by liquid having therapeutic benefit may increase the overall amount of therapy given to the wound bed. This may effectively result in increased time of new beneficial therapy in a 24-hour span where previously only suction pressure therapy existed. The net result is the even, regular addition of many new extra hours of beneficial therapy interspersed between suction pressure therapy that may accelerate healing through synergistic effects. Because chronic wound healing may be extremely protracted, the ability to add additional therapy each and every day—without reducing the duration of the fundamental pressure therapy—may serve as a de novo creation of additional synergies that may accelerate healing.

For example, consider a pressure cycle having a 6-minute period with pressure p₀ within an enclosed space at pₘᵢₙ for 4 minutes (⅔ of the period of the pressure cycle) and the pressure p₀ relieved to pₘₐₓ for 2 minutes (⅓ of the period of the pressure cycle). In this example, the relief of the pressure p₀ from pₘᵢₙ to pₘₐₓ using fluid with O₂ concentration greater than atmospheric air results in about 2 minutes per cycle of topical oxygen therapy at maximum pressure pₘₐₓ, totaling the equivalent of 8 hours per day of topical oxygen therapy at maximum pressure pₘₐₓ. Furthermore, even during the active suction phase of the therapy where pressure p₀ within the enclosed space is pₘᵢₙ, the oxygen that persists from the previous relief of the pressure p₀ from pₘᵢₙ to pₘₐₓ using fluid with O₂ concentration greater than atmospheric air will continue to oxygenate the wound bed and inhibit bacterial growth. This may result in delivering the benefit of oxygen to the wound bed around the clock.

As a second example, consider a pressure cycle that has a 6-minute period with pressure p₀ within an enclosed space at pₘᵢₙ for 3 minutes and the pressure p₀ relieved to pressure pₘₐₓ for 3 minutes per cycle (½ of the period of the pressure cycle) using fluid with O₂ concentration greater than atmospheric air. This results in delivery of topical oxygen therapy to the wound bed at pressure pₘₐₓ totaling 12 hours per day, in this second example. In this second example, pₘₐₓ may be approximately equal to ambient pressure p_{amb}. Therefore, towards the latter stages of healing of the wound bed when pressure pₘᵢₙ is less needed, the duration of topical oxygen at pressure pₘₐₓ can be correspondingly increased.

Such O₂ enrichment at pressure pₘₐₓ provided to the wound bed may be beneficial because the O₂ enrichment is [1] under a favorable concentration gradient, [2] at a favorable pressure gradient that does not impede baseline arteriole perfusion (such as between 20-60 mm Hg, but may be higher for brief durations), and [3] during a period of relative reflex hyperemia in regions of tissue where capillaries may have been collapsed under suction. The result is the maximum absorption and uptake of oxygen under increased-flow condition.

Additionally, in aspects wherein the fluid-tight enclosed space provides a hyperbaric condition (p₀>p_{amb} with enhanced O₂ concentration), the amplitude and period of the pressure p₀ may additionally serve to provide a form of external pulsation of pressurized O₂, with beneficial circulatory effect akin in some respects to providing external CPR to the wound bed.

By using fluid with O₂ concentration greater than that found in atmospheric air during at least portions of the pressure cycle, the resulting O₂ enrichment may resuscitate the hypoxic wound cells, may sustain the revived cells in cell division and collagen synthesis, may inhibit the growth of anaerobic bacteria, may enhance the efficacy of antibiotics, and may enhance survival of skin grafts.

In various aspects, every nth pressure cycle (where n is any suitable number such as 2 through 60 or even 120 or more) is relieved with liquid such as, for example, saline solution, proteolytic enzyme solution, biofilm degradation solution, antibiotic lavage, amniotic fluid, platelet-enriched plasma, antibiotic, anesthetic, or other liquid having therapeutic benefits.

In various aspects, the apparatus and related methods of wound therapy may include distending periodically portions of the wound bed into the enclosed space by evacuating fluid from the enclosed space and retracting the distended portions of the wound bed from the enclosed space by inputting fluid into the enclosed space and thereby varying the pressure p₀ within the enclosed space periodically over the pressure cycle. In some aspects, the enclosed space may be defined, at least in part, by a wound interface sufficiently deformation resistant to accommodate distention of the wound bed into the enclosed space when the pressure p₀ within the enclosed space is less than ambient pressure p_{amb}.

In various aspects, the apparatus and related methods of wound therapy include absorbing exudate from the wound bed intermittently by periodically bringing a pad positioned within the wound interface into fluid communication with at least a portion of the wound bed during at least a portion of the step of distending periodically portions of the wound bed into the enclosed space, the pad adapted to absorb exudate from the wound bed.

Ambient pressure p_{amb}, as used herein, refers to the pressure in a region surrounding the wound therapy apparatus. Ambient pressure p_{amb}, for example, may refer to atmospheric pressure, hull pressure within an aircraft where the wound therapy apparatus is being utilized, or pressure maintained generally within a building or other structure where the wound therapy apparatus is being utilized. Ambient pressure p_{amb} may vary, for example, with elevation or weather conditions. Pressure pₘᵢₙ refers to the minimum pressure achieved within the enclosed space of the wound interface, and periodically varying of pressure p₀, pressure variation, varying pressure, and similar term refer to changes of pressure p₀ within the enclosed space over time, in various aspects. Pressure pₘₐₓ refers to the maximum pressure achieved within the enclosed space of the wound therapy apparatus.

In various aspects, the term fluid-tight or related terms, as used herein, means sufficiently leak-resistant to allow insufflation or vacuum suction to create pressure p₀ within the enclosed space that may be above or below ambient pressure p_{amb}. The term fluid-tight means sufficiently leak-resistant to substantially retain fluids including both gasses and liquids within the enclosed space other than by controlled fluid communication through one or more lumen that fluidly communicate through the wound interface with the enclosed space, in certain aspects. In certain aspects, fluid tight means sufficiently leak-resistant to maintain pressure p₀ within the enclosed space that may be above or below ambient pressure p_{amb}.

At least one of the one or more lumen may fluidly communicate with the pad to allow transfer of exudate from the pad. Optionally, at least one of the one or more lumen may be fluidly engaged in monitoring directly or indirectly intra-enclosed space parameters such as pressure, temperature, pH, oxygen concentration, blood flow, etc. to effect improved therapy.

Exudate, as used herein, includes, for example, proteinaceous liquids exuded from the wound bed, along with various plasma and blood components. Exudate may also include other liquids used in treating the wound bed.

Fluid, as used herein, includes liquid(s), gas(ses), and combinations thereof. Gas may include, for example, oxygen, oxygen enriched air, humidity, nitric oxide, other gas, and combinations thereof. Fluid may include exudate. Humidity, as used herein, includes water vapor and mist.

As used herein the terms distal and proximal are defined from the point of view of a healthcare provider. A distal portion of the wound therapy apparatus is oriented toward the patient while a proximal portion of the wound therapy apparatus is oriented toward the physician. For example, a distal portion of the wound interface may be closest to the patient while a proximal portion of the wound interface may be closest to the physician when said wound interface is being used to treat the patient.

As used herein, a wound interface that is deformation resistant forms an enclosure that resists collapse and substantially maintain its shape including defining an enclosed space within sufficient to draw the wound bed towards the enclosed space up to the point of occupying that enclosed space when subjected to pressure p₀≤p_{amb}, in various aspects. In some aspects, at least portions of the wound interface that define the enclosed space may be essentially rigid. The wound interface, in various aspects, is sufficiently deformation resistant to remain sealingly secured to skin and fluid-tight over pressure range pₘᵢₙ≤p₀≤pₘₐₓ.

Massaging of the wound bed via pressure variations, including rhythmic distortion of the wound bed volume, may be accompanied by fluxes of increased blood flow. The terms massage, massaging, rhythmic distortion, tissue deformation, distention of wound bed may be used interchangeably in this disclosure to refer to the general process of subjecting the wound bed to pressure fluctuations and the resultant changes in the wound bed, including blood flow, oxygenation, cellular tension and other changes. The surges of increased blood flow proximate the wound bed may bring increased nutrients, reduce infection and inflammation, and confer other beneficial effects that may promote healing of the wound bed. Massaging of the wound bed may promote the removal of exudate from the interstitial space of the wound bed to exit the wound crater. This may reduce capillary compression secondary to edema and improve the microcirculation to and within the wound. At least one of the one or more ports may fluidly communicate with the pad to allow transfer of exudate from the pad. Optionally, at least one of the one or more ports may be fluidly used for monitoring directly or indirectly intra-enclosed space parameters such as pressure, temperature, humidity, pH, tissue oxygenation level, blood flow, etc. to effect improved therapy.

The methods of wound therapy include, in various aspects, providing a therapy regimen to the wound bed, the therapy regimen comprising delivering consecutively a number of pressure cycles of a pressure p₀ within the enclosed space, each pressure cycle comprising a pressure range pₘᵢₙ≤p₀≤pₘₐₓ.

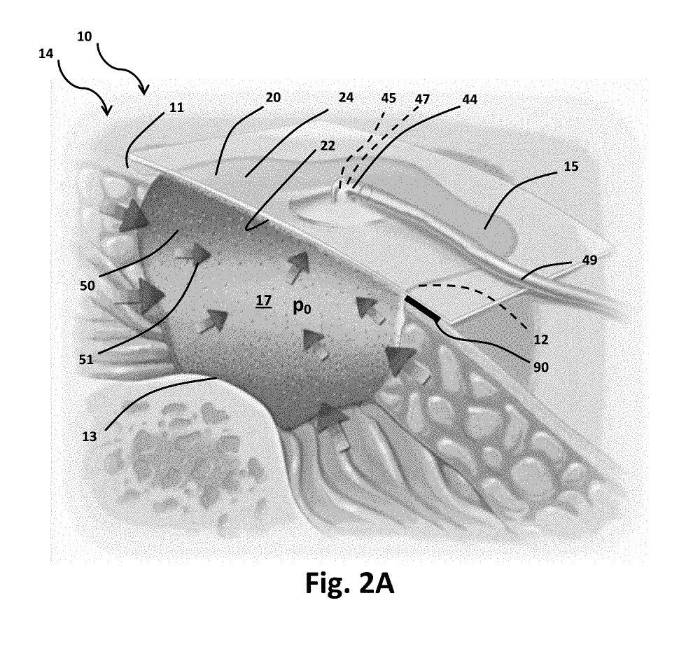

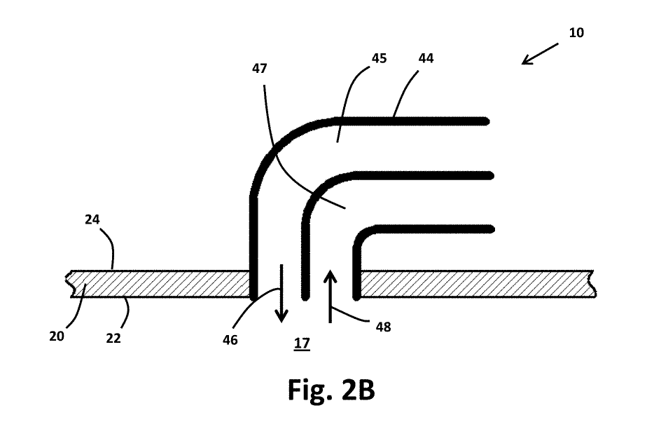

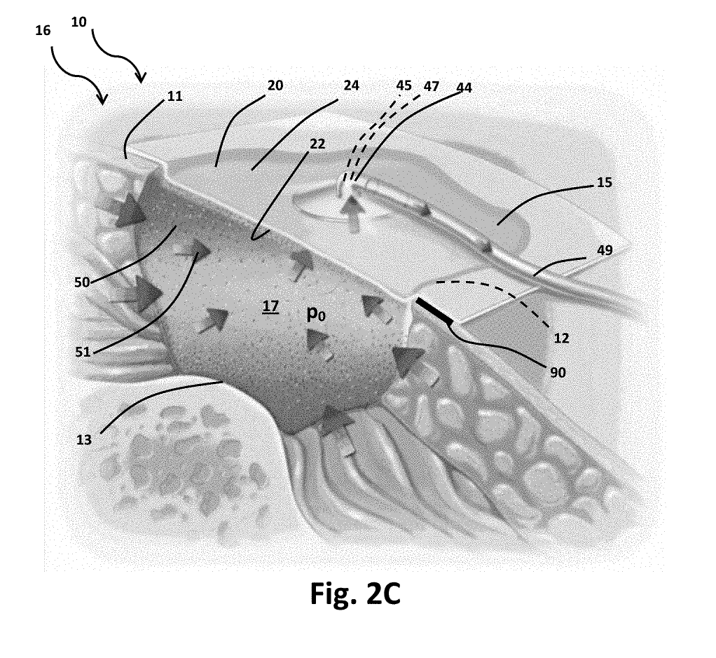

An exemplary implementation of a wound therapy apparatus 10 is illustrated in FIGS. 2A, 2B, and 2C. As illustrated in FIG. 2A, wound therapy apparatus 10 includes wound interface 15, and wound interface 15 includes sheet 20 attached to skin surface 11 by adhesive 90 to enclose wound bed 13 at skin surface 11, with the entirety of wound boundary 12 covered by sheet 20. Distal side 22 of sheet 20 faces wound bed 13 and adhesive 90 on at least portions of distal side 22 secures sheet 20 to skin surface 11 thereby defining enclosed space 17, as illustrated. Enclosed space 17 includes the region between sheet 20 and wound bed 13 sealingly enclosed by securement of sheet 20 to skin 11, as illustrated. Dressing 50 is packed into wound bed 13 and covered by sheet 20, as illustrated, so that dressing 50 lies within enclosed space 17.

As illustrated in FIG. 2B, port 44 defines lumen 45, 47 passing between distal side 22 of sheet 20 and proximal side 24 of sheet 20 for fluid communication with enclosed space 17. Tubing 49, as illustrated in FIG. 2A, is coupled to port 44 for fluid communication with enclosed space 17 via lumen 45, 47. Tubing, as used herein, includes, for example, hoses, pipes, as well as valves, fittings, chambers, pressure vessels, sumps, and reservoirs. Dressing 50 may be, for example, cotton gauze or open-cell foam made from polyvinyl alcohol or polyurethane and lumen 47 may be in fluid communication with dressing 50 to withdraw exudate from dressing 50.

Sheet 20 may be flexible to deform in conformance to the wound bed 13 proximate skin surface 11 when pressure p₀ within enclosed space 17 is less than p_{amb}. Sheet 20 may be formed of polymer with adhesive disposed upon at least portions of distal side 22 to affix the sheet 20 to the skin surface 11 around wound boundary 12 at skin surface 11. While sheet 20 may be referred to as impermeable, it is understood that the permeability of the sheet may be generally limited to transpiration (to allow the skin to ‘breathe’) and not the ready passage of fluids.

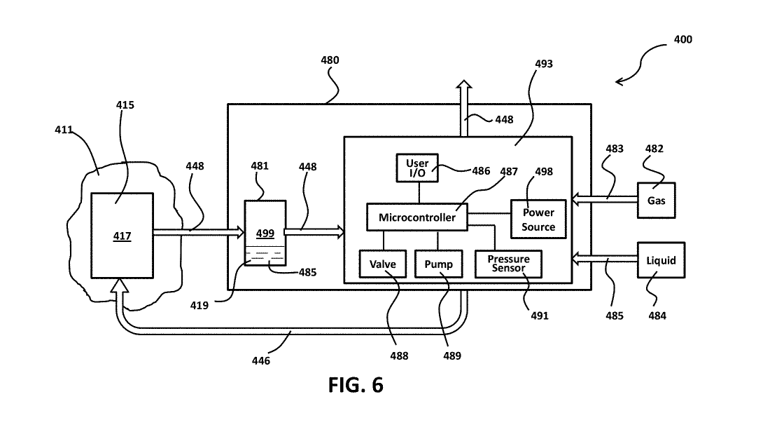

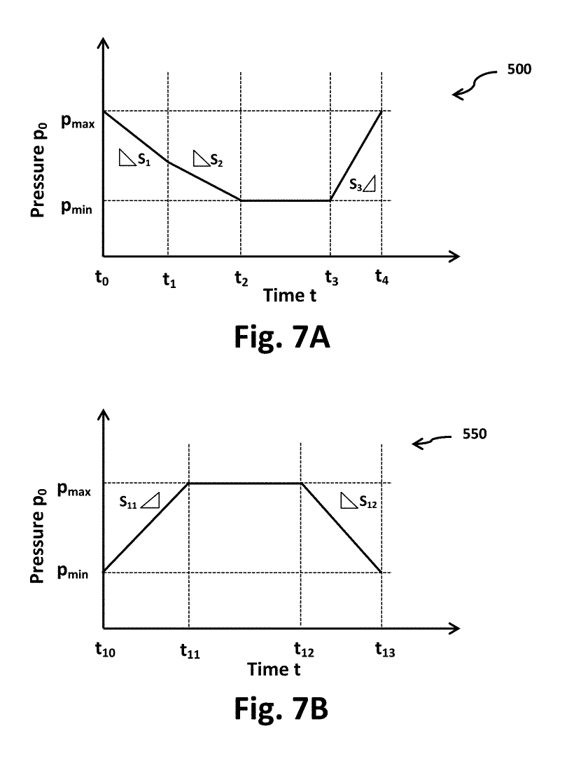

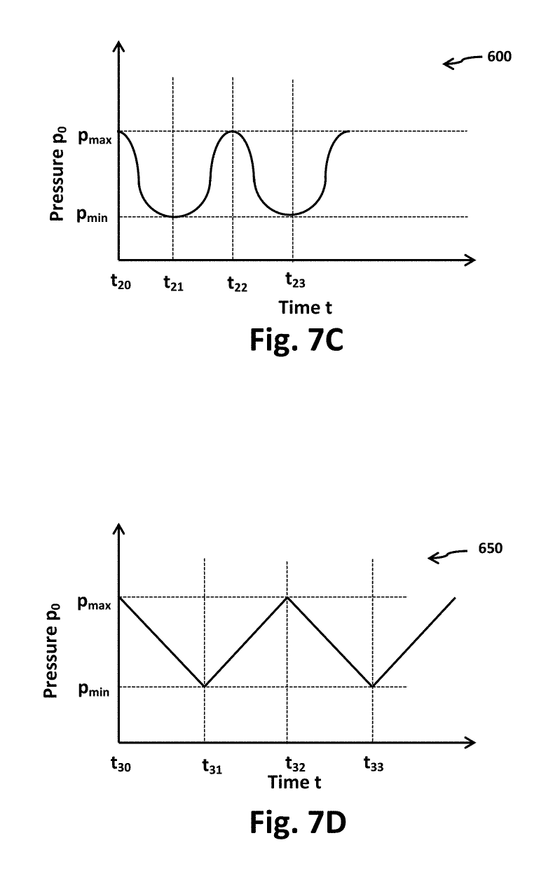

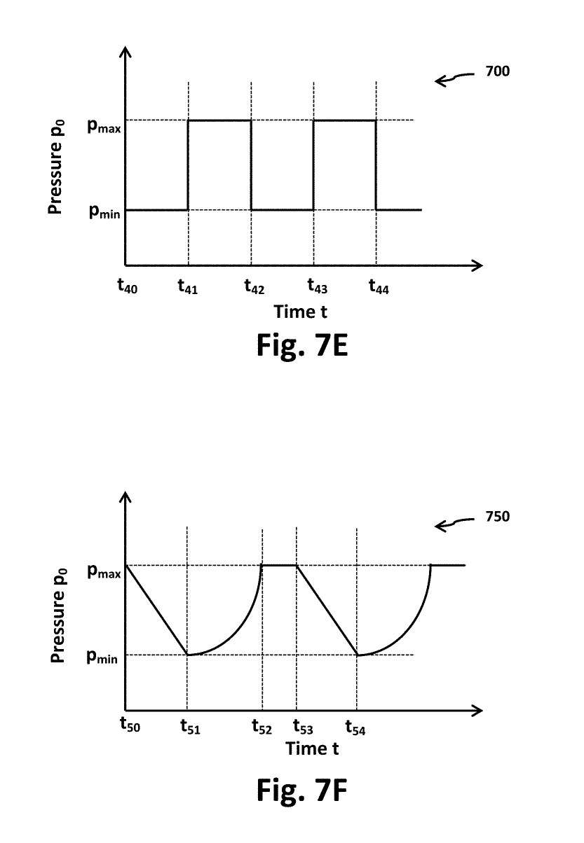

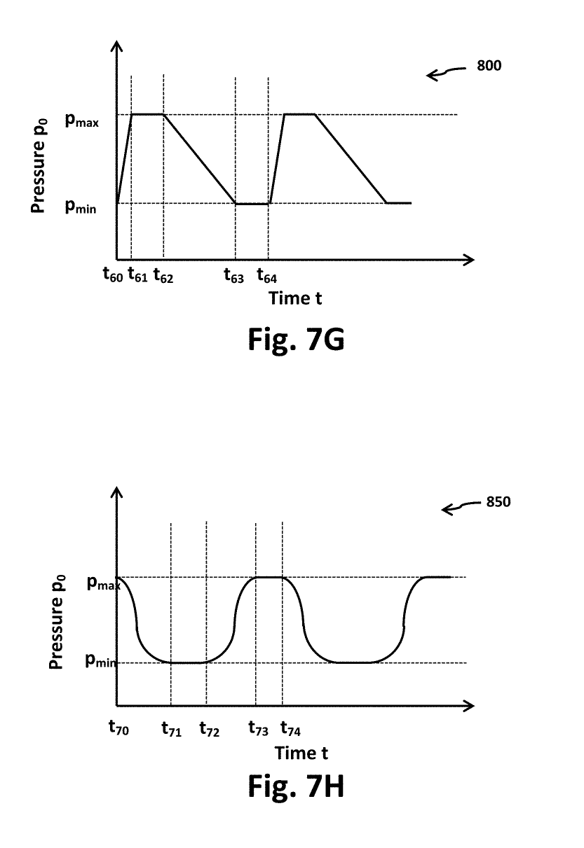

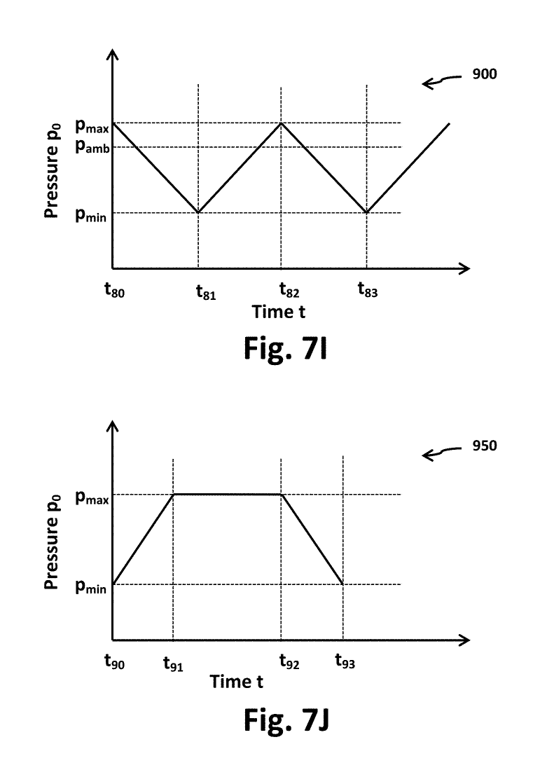

In operation, wound therapy apparatus 10 may be varied periodically between first stage of operation 14 illustrated in FIG. 2A and second stage of operation 16 illustrated in FIG. 2C by varying pressure p₀ within enclosed space 17 periodically according to a pressure cycle, such as pressure cycle 500, 550, 600, 650, 700, 750, 800, 850, 900, 950 (see FIGS. 7A, 7B, 7C, 7D, 7E, 7F, 7G, 7H, 7I, 7J, respectively). During the pressure cycle, the pressure p₀ within enclosed space 17 may vary over the pressure range pₘᵢₙ≤p₀≤pₘₐₓ where pₘᵢₙ is the minimum pressure over the pressure cycle and pₘₐₓ is the maximum pressure over the pressure cycle. The minimum pressure may, for example, generally range from about pₘᵢₙ=p_{amb}−80 to about p_{amb}−175 mm Hg, and the maximum pressure pₘₐₓ may range, for example, from about ambient pressure p_{amb} to about p_{amb}+40 mm Hg. In some implementations, the maximum pressure pₘₐₓ may be slightly less than ambient pressure pₐₘ, for example, generally within the range of p_{amb}−5 mm Hg to p_{amb}−20 mm Hg. Lumen 45, 47 of port 44 may fluidly communicate with a controller, such as controller 480 of wound therapy apparatus 400 (see FIG. 6), and the controller may input the input fluid 46 into enclosed space 17 or withdraw output fluid 48 from enclosed space 17 to vary the pressure p₀ within enclosed space 17 over the pressure range pₘᵢₙ≤p₀≤pₘₐₓ.

In first stage of operation illustrated in FIG. 2A, pressure p₀=pₘₐₓ≈p_{amb}. As wound therapy apparatus 10 is varied from first stage of operation 14 to second stage of operation 16, which is illustrated in FIG. 2C, the pressure p₀ within enclosed space 17 decreases from pₘₐₓ with p₀→pₘᵢₙ so that pressure p₀≈pₘᵢₙ at second stage of operation 16. Output fluid 48, including air or other gasses within enclosed space 17, is evacuated from enclosed space 17 through lumen 47, illustrated in FIG. 2B, by suction applied to lumen 47 to place wound therapy apparatus 10 in second stage of operation 16 with pressure p₀≈pₘᵢₙ. Output fluid 48 may include exudate 51 that may be withdrawn from wound bed 13 or from dressing 50 through lumen 47. Exudate 51 is illustrated as migrating from wound bed 13 through dressing 50 toward lumen 47 using large black arrows, in FIGS. 2A, 2C. Wound therapy apparatus 10 may be maintained in a condition of stasis at second stage of operation 16 for some period of time with essentially no inflow of input fluid 46 or withdrawal of output fluid 48 other than exudate 51 so that no flow of fluid passes from lumen 45 through enclosed space 17 and then out of lumen 47 in the condition of stasis when second stage of operation 16 is in the condition of stasis.

As wound therapy apparatus 10 is varied from second stage of operation 16 to first stage of operation 14, the pressure p₀ within enclosed space 17 is increased from pₘᵢₙ with p₀→pₘₐₓ so that pressure p₀≈p_{amb} at first stage of operation 14, in this implementation. Note that pₘₐₓ>p_{amb} in other implementations of first stage of operation 14. Input fluid 46 is input into enclosed space 17 through lumen 45, as illustrated in FIG. 2B, to place wound therapy apparatus 10 into first stage of operation 14 from second stage of operation 16. In various implementations, input fluid 46 has an O₂ concentration greater than that found in atmospheric air. In various implementations, input fluid 46 may be O₂ with humidity. In various implementations, input fluid 46 may be O₂ in combination with other gasses. In various implementations, input fluid 46 may include, for example, air, oxygen, air with enhanced oxygen concentration, nitric oxide, nitrogen, humidity, other therapeutic gasses or inert gasses, and combinations thereof.

Periodic variation of pressure p₀ generally over the pressure range pₘᵢₙ≤p₀≤pₘₐₓ may induce corresponding periodic surges of fresh blood flow into the wound bed that provides, for example, nutrients, immune factors, and oxygen. Introducing oxygen O₂ at concentrations greater than those of atmospheric air may provide additional benefits in wound therapy. Input fluid 46 is input into enclosed space 17 sequentially with respect to the withdrawal of output fluid 48 from enclosed space 17, as pressure p₀ is varied periodically over the pressure range pₘᵢₙ≤p₀≤pₘₐₓ, in exemplary wound therapy apparatus 10. Input of input fluid 46 does not occur simultaneously with withdrawal of output fluid 48. In various implementations, the pressure p₀ may be varied generally over the pressure range pₘᵢₙ≤p₀≤pₘₐₓ several times per hour, for example, approximately once about every 5 minutes or once about every 6 minutes.

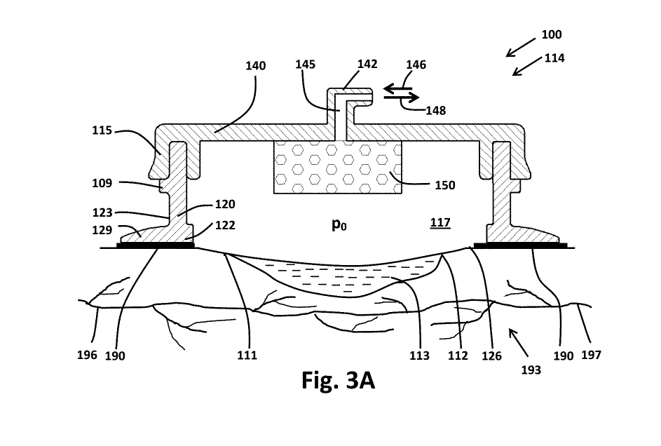

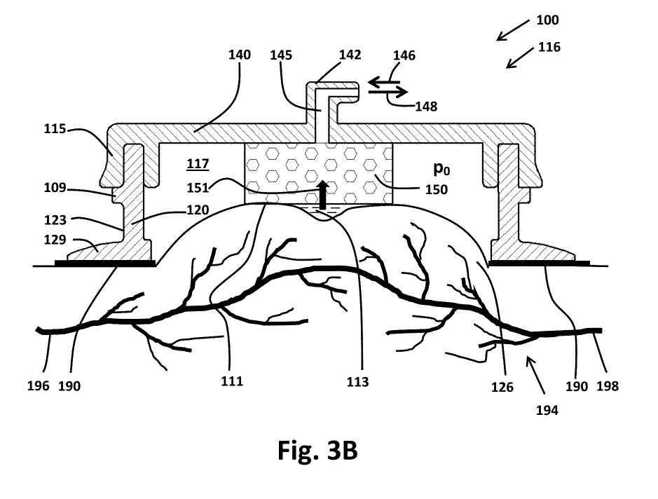

FIGS. 3A and 3B illustrate exemplary wound therapy apparatus 100 at exemplary first stage of operation 114 and at exemplary second stage of operation, respectively. As illustrated in FIGS. 3A and 3B, wound therapy apparatus 100 includes wound interface 115 that is deformation resistant and defines enclosed space 117 that is fluid-tight when wound interface 115 is engaged with skin surface 111 to enclose wound bed 113 at skin surface 111. Wound interface 115, as illustrated in FIG. 3A, includes cover 140 slidably sealingly frictionally removably engaged with base 120. Cover 140 may include at least transparent portions to allow visual inspection of wound bed 113 or pad 150 though cover 140. Base 120 may include flange 109 formed entirely around an outer perimeter of base 120 that may provide structural support or sealing surface that cooperates with cover 140, as illustrated. In other implementations, cover 140 and base 120 may be formed as a unitary structure, or cover 140 may be engaged hingedly or engaged in other ways with base 120.

While wound interface 115 is illustrated as cylindrical in shape enclosing a circular region of skin surface 111, it should be understood the wound interface, such as wound interface 115, may assume other geometric shapes to enclose other geometrically shaped regions of skin 111 such as rectangular, polygonal, or ovoid, to enclose various shaped wounds or regions over skin surface 111, in various other implementations. For example, the wound interface 115 may be ovoid shaped and low profile in shape to enclose a linear incision, for example, as may surround a wound bed resulting from a Caesarian section. The wound interface may be ovoid and higher profile to enclose the breasts following breast augmentation or reconstructive breast surgery following mastectomy.

Base 120, in this implementation, includes flange 129 around the entire perimeter of outer side 123 of base 120 generally at distal end 122 of base 120. Flange 129 is secured to skin surface 111 by adhesive 190, as illustrated in FIGS. 3A and 3B. Flange 129 is secured sealingly to the skin surface 111 around the entire perimeter of base 120 to form fluid-tight enclosed space 117, as illustrated, and wound boundary 112 is enclosed within enclosed space 117. Flange 129 may be designed by thickness and/or polymeric material to be soft and conformable to enable sealing of wound interface 115 over a wound 113 in a fluid-tight manner while distributing forces on wound interface 115 from pressure p₀ within enclosed space 117 over the skin surface 111.

Adhesive layer 190 may optionally extend over portions of skin surface 111 to include all skin surface under and proximate to flange 129 at distal end 122 including skin surface proximate wound bed 113. When the adhesive layer 190 is a medically suitable member of the cyanoacrylate class, such as N-butyl-2-cyanoacrylate (Histoacryl Blue), or octyl-2-cyanoacrylate (Dermabond), the layer of water-resistant adhesive coating over the peri-wound skin surface serves the additional function of protecting the normal skin from maceration, secondary to prolonged exposure to other fluids, such as exudate, proteolytic enzyme soaks or saline lavages, etc. Other medical adhesives, for example, acrylic, silicone and hydrocolloid may be used as adhesive 190 to secure wound interface 115 to the skin surface 111. Adhesive 190 may comprise combinations of adhesives, in various implementations. Other securements such as straps with hook-and-loop-type fasteners may also be employed in various other implementations to secure, at least in part, wound interface 115 to the skin surface 111. Base 120 of wound interface 115 may be formed of any of various medical polymers including polystyrene or polypropylene.

Port 142, which is located about wound interface 115, is in fluid communication with enclosed space 117 via lumen 145. Port 142 may be configured for attachment to tubing for the communication of fluids via enclosed space 117 through lumen 145. A pad 150 may be deployed within enclosed space 117 to absorb exudate from wound bed 113, and the pad 150 may be in fluid communication with port 142 to allow withdrawal of exudate 151 from wound bed 113 through the pad and thence through port 142.

Pad 150 may be formed of absorbent material(s) that absorb exudate 151 including open-cell foam composed, for example, of polyvinyl alcohol (PVA), polyurethane or other polymer foam. Pad 150 may be formed of various woven or non-woven fibers such as sodium carboxymethyl, cellulose hydrofiber (Aquacel), or knitted fibers with hydrophobic polyester fiber predominant on outer surface and hydrophilic nylon fibers predominantly on the inside to serve as a conduit to fluid transfer. In such implementations, the hydrophobic polyester fiber wicks away liquid and prevents moisture buildup and, thus, maceration of tissue with which pad 150 may be in contact.

Input fluid 146 may be input into enclosed space 117 via lumen 145 of port 142, as indicated by the arrow in FIGS. 3A, 3B, for example, to regulate, at least in part, the pressure p₀ within enclosed space 117, to control the composition of the gaseous fluids within enclosed space 117, or for various therapeutic purposes. Input fluid 146, for example, may be input into enclosed space 117 to increase the pressure p₀ within enclosed space 117. In various implementations, input fluid 146 has an O₂ concentration greater than that found in atmospheric air. In various implementations, the input fluid 146 may be essentially O₂ or O₂ of medical purity. In various implementations, input fluid 146 may be O₂ with humidity. In various implementations, the input fluid 146 may include O₂ in combination with other gasses including humidity. In various implementations, the input fluid 146 may include nitric oxide in 200 ppm to 1000 ppm dilution. Output fluid 148, which may include gas, liquid, or combinations of gas and liquid within enclosed space 117, may be withdrawn from enclosed space 117 through lumen 145 of port 142, as illustrated, for example to decrease the pressure p₀ within enclosed space 117. Output fluid 148 may include exudate, such as exudate 151, from wound bed 113 or from pad 150. Fluids 146, 148 may include liquids that may have various therapeutic purposes.

In operation, wound therapy apparatus 100 may be periodically varied between first stage of operation 114 illustrated in FIG. 3A and second stage of operation 116 illustrated in FIG. 3B by consecutive withdrawal of output fluid 148 from enclosed space 117 and input of input fluid 146 into enclosed space 117 via port 142. The pressure p₀ within enclosed space 117 may be varied with respect to time t according to a pressure cycle, such as pressure cycle 500, 550, 600, 650, 700, 750, 800, 850, 900, 950 (see FIGS. 7A, 7B, 7C, 7D, 7E, 7F, 7G, 7H, 7I, 7J, respectively), between first stage of operation 114 and second stage of operation 116. Input fluid 146 is input into enclosed space 117 sequentially with the withdrawal of output fluid 148 from enclosed space 117, as pressure p₀ is varied, for example, between first stage of operation 114 and second stage of operation 116, in exemplary implementation of a wound therapy apparatus 100. A controller, such as controller 480 of wound therapy apparatus 400, may be in fluid communication with lumen 145 of port 142 to input the input fluid 146 into enclosed space 117 and to withdraw output fluid 148 from enclosed space 117 in order to vary the pressure p₀ within enclosed space 117, for example, according to pressure cycle 500, 550, 600, 650, 700, 750, 800, 850, 900, 950.

During the pressure cycle, the pressure p₀ within enclosed space 117 may vary over the pressure range pₘₘ≤p₀≤pₘₐₓ where pₘᵢₙ is the minimum pressure over the pressure cycle and pₘₐₓ is the maximum pressure over the pressure cycle. For example, the minimum pressure pₘᵢₙ≈p_{amb}−30 mm Hg and the maximum pressure pₘₐₓ≥p_{amb}. The minimum pressure may be, for example, pₘᵢₙ≈p_{amb}−130 mm Hg. The minimum pressure may be, for example, pₘᵢₙ≈p_{amb}−90 mm Hg. The minimum pressure may be, for example, generally within the pressure range (p_{amb}−130 mm Hg)≤pₘᵢₙ≤(p_{amb} 90 mm Hg). The minimum pressure pₘᵢₙ may be generally within the pressure range (p_{amb}−90 mm Hg)≤pₘᵢₙ<p_{amb}. In various implementations, the periodic variation of the pressure p₀ may be generally within the pressure range pₘᵢₙ≤p₀≤pₘₐₓ where pₘₐₓ>p_{amb}. For example, pₘₐₓ≈(p_{amb}+40 mm Hg). In various implementations, pₘₐₓ≈p_{amb}. In various implementations, pressure pₘₐₓ may be slightly less than ambient pressure p_{amb}, for example, by −10 mm Hg or −20 MMHg.

At a particular time during the pressure cycle the pressure p₀ may be generally constant throughout enclosed space 117, so that the entirety of wound bed 113 is exposed to pressure p₀, and, thus, no pressure gradient is created about wound bed 113 that may, for example, decrease blood flow proximate the wound boundary 112.

At exemplary first stage of operation 114, as illustrated in FIG. 3A, the pressure p₀≈pₘₐₓ within enclosed space 117. Wound bed 113 is in a baseline state 193, and wound bed 113 is in spaced relation with pad 150 so that wound bed 113 does not contact pad 150. As illustrated in FIG. 3A, wound interface 115 defines entry 126 into enclosed space 117, and the portions of wound bed 113 enclosed by enclosed space 117 may generally lie outside entry 126 in baseline state 193. Capillary 196, which is proximate wound bed 113, is in a baseline undilated condition 197 and conveys a baseline quantity of blood to wound bed 113 when wound bed 113 is in baseline state 193 at first stage of operation 114 illustrated in FIG. 3A.

At exemplary second stage of operation 116 of wound therapy apparatus 100, as illustrated in FIG. 7B, enclosed space 117 is evacuated, in part, by withdrawal of output fluid 148 from enclosed space 117 through lumen 145 of port 142 so that the pressure p₀ within enclosed space 117 is equal to pressure pₘᵢₙ which is less than ambient pressure p_{amb} pₘᵢₙ<p_{amb}) by an amount sufficient to cause least portions of wound bed 113 to be distended into enclosed space 117 through entry 126 in distended state 194, as illustrated in FIG. 3B. At least portions of wound bed 113 bias against pad 150 at second stage of operation 116, as illustrated in FIG. 3B. Pad 150 may thus absorb exudate 151 from wound bed 113 at second stage of operation 116. Pad 150 fluidly communicates with lumen 145 of port 142 so that exudate 151 may be withdrawn from pad 150 through lumen 145 of port 142 as at least a portion of output fluid 148 via external suction applied to port 142, as illustrated. Capillary vessels proximate the wound bed, such as capillary 196, may be in a dilated state 198 when wound bed 113 is in distended state 194 at second stage of operation 116, as illustrated in FIG. 3B. Wound therapy apparatus 100 may be maintained at second stage of operation 116 for some time. Essentially no input of input fluid 146 or withdrawal of output fluid 148 other than exudate 151 may occur so that essentially no fluid flows through enclosed space 117 while wound therapy apparatus is being maintained at either first stage of operation 114 or second stage of operation 116.

Wound therapy apparatus 100 may be varied periodically between first stage of operation 114 and second stage of operation 116 by varying pressure p₀ within enclosed space 117 periodically generally over the pressure range pₘᵢₙ≤p₀≤pₘₐₓ to distend wound bed 113 into enclosed space 117 in distended state 194 and to release wound bed 113 from distention into enclosed space 117 back to baseline state 193, respectively, thereby massaging wound bed 113. In various implementations, pₘᵢₙ<p_{amb} and p_{amb}≤pₘₐₓ. The maximum pressure pₘₐₓ may be greater than ambient pressure p_{amb}, may be generally equal to ambient pressure p_{amb}, or may be less than ambient pressure p_{amb}, in various implementations. Periodically releasing wound bed 113 from contact with pad 150 by altering wound therapy apparatus 100 from second stage of operation 116 to first stage of operation 114 may prevent wound bed 113 from becoming attached to pad 150. Granulation tissue of wound bed 113 will not have time to grow into pad 150, and, in turn, will not become disrupted when pad 150 or wound interface 115 is replaced. This may be an important benefit over current NPWT devices where the granulation tissue is sucked into the dressing and then painfully disrupted by dressing changes.

The wound interface 115 may be sufficiently deformation resistant to remain fluid-tight when pressure p₀=pₘᵢₙ, thereby allowing wound bed 113 to be distended into enclosed space 117 and released from distented state 194 back to relaxed state 193. The wound interface 115 may be sufficiently deformation resistant to maintain enclosed space 117 with entry 126 when pressure p₀=pₘᵢₙ, thereby allowing wound bed 113 to be distended into enclosed space 117 in distended state 194 and released from distended state 194 back to relaxed state 193. In some implementations, wound interface 115 may be sufficiently rigid to not deform over the pressure range pₘᵢₙ≤p₀≤pₘₐₓ.

The periodic variation of pressure p₀ generally over the pressure range pₘᵢₙ≤p₀≤pₘₐₓ and corresponding alterations of the wound bed 113 between relaxed state 193 and distended state 194 may induce corresponding periodic surges of fresh blood flow into the wound bed that provides, for example, nutrients, immune factors, and oxygen. Such distention including deformation and stretching of tissues surrounding the wound bed has been found to stimulate fibroblast differentiation and wound healing (cf. Saxena, V. et. al., Vacuum Assisted Closure: Microdeformation of Wound and Cell Proliferation. Amer. Soc. Plastic Surg. 1086-1096, October 2004). Such periodic variations of pressure p₀ generally over the pressure range pₘᵢₙ≤p₀≤pₘₐₓ and corresponding alterations of the wound bed 113 between relaxed state 193 and distended state 194 may occur over a period lasting several minutes, such as about 5 minutes or about 6 minutes, or may occur over time periods up to an hour or two, in various implementations.

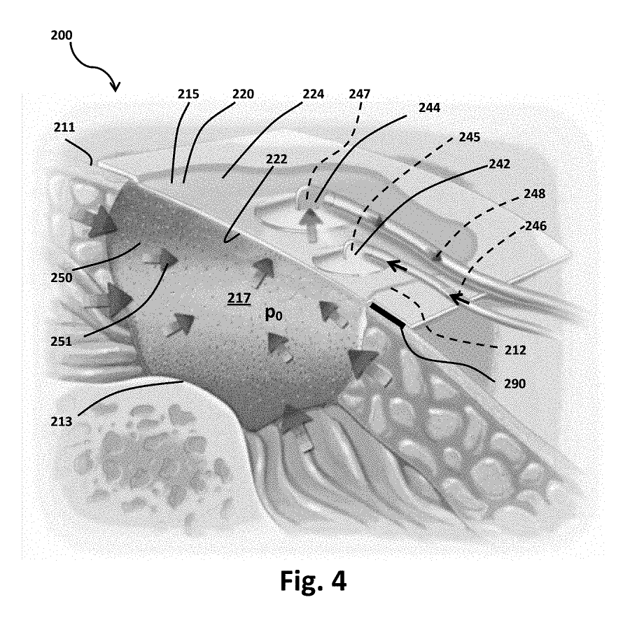

An exemplary implementation of a wound therapy apparatus 200 is illustrated in FIG. 4. As illustrated in FIG. 4, wound therapy apparatus 200 includes wound interface 215, and wound interface 215 includes sheet 220 attached to skin surface 211 to enclose wound bed 213 at skin surface 211, with the entirety of wound boundary 212 covered by sheet 220. Sheet 220 may be made of a single layer of material, in some implementations, or may be made of several layers of material, in other implementations. Distal side 222 of sheet 220 faces wound bed 213, and adhesive 290 on distal side 222 secures sheet 220 to skin surface 211 thereby defining portions of enclosed space 217, as illustrated. Enclosed space 217 includes at least portions of wound bed 213, as illustrated. Dressing 250 is packed into wound bed 213 and covered by sheet 220, as illustrated, within at least portions of enclosed space 217. As illustrated in FIG. 4, ports 242, 244 are in fluid communication with enclosed space 217 between distal side 222 of sheet 220 and proximal side 224 of sheet 220 by lumen 245, 247, respectively. Input fluid 246 may be input into enclosed space 217 via lumen 245 of port 242 and output fluid 248 including exudate 251 may be withdrawn from enclosed space 217 via lumen 247 of port 244 as pressure p₀ within enclosed space 217 is periodically varied with a pressure cycle generally over the pressure range pₘᵢₙ≤p₀≤pₘₐₓ where pₘᵢₙ is the minimum pressure over the pressure cycle and pₘₐₓ is the maximum pressure over the pressure cycle. The pressure cycle of pressure p₀ within enclosed space 217 may be, for example, pressure cycle 500, 550, 600, 650, 700, 750, 800, 850, 900, 950 (see FIGS. 7A, 7B, 7C, 7D, 7E, 7F, 7G, 7H, 7I, 7J, respectively). A controller, such as controller 480 of wound therapy apparatus 400, may be in fluid communication with lumen 245 of port 242 and lumen 247 of port 244 to input the input fluid 246 into enclosed space 217 and to withdraw output fluid 248 from enclosed space 217, respectively, in order to vary the pressure p₀ within enclosed space 217, for example, according to pressure cycle 500, 550, 600, 650, 700, 750, 800, 850, 900, 950.

Input of input fluid 246 into enclosed space 217 via lumen 245 and withdrawal of output fluid 248 from enclosed space 217 via lumen 247 may be sequential with one another, meaning input fluid 246 is not input into enclosed space 217 simultaneously with withdrawal of output fluid 248 from enclosed space 217, in this implementation. Input fluid 246 may be being input into enclosed space 217 while no output fluid 248 is being withdrawn from enclosed space 217, output fluid 248 may be being withdrawn from enclosed space 217 while no input fluid 246 is being input into enclosed space 217, or no input fluid 246 is being input into enclosed space 217 and no output fluid 248 is being withdrawn from enclosed space 217, in various implementations.

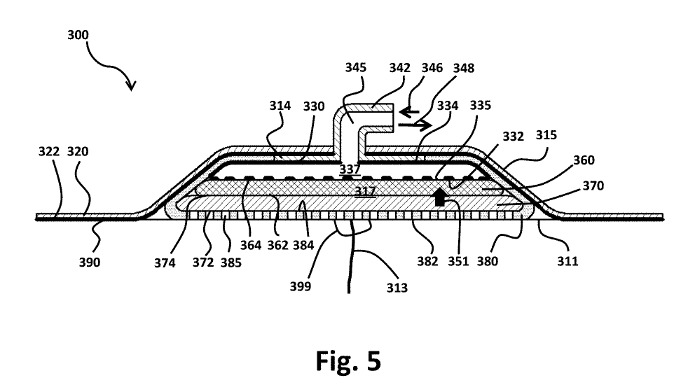

An exemplary implementation of a wound therapy apparatus 300 is illustrated in FIG. 5. As illustrated in FIG. 5, wound therapy apparatus 300 includes wound interface 315, and wound interface 315 includes member 320 with adhesive 390 coated on at least portions of distal surface 322 of member 320 to secure member 320 to skin surface 311. When distal surface 322 is secured to skin surface 311, wound interface 315 encloses wound bed 313 at skin surface 311 within enclosed space 317, as illustrated. As illustrated in FIG. 5, flange 314 of port 342 secures port 342 to member 320 for fluid communication with enclosed space 317 by lumen 245. Input fluid 346 may be input into enclosed space 317 via lumen 345 of port 342 and output fluid 348 may be withdrawn from enclosed space 317 via lumen 345 of port 342, for example, as target pressure p₀ within enclosed space 317 is periodically varied with a pressure cycle generally having a pressure range pₘᵢₙ≤p₀≤pₘₐₓ where pₘᵢₙ is the minimum pressure over the pressure cycle and pₘₐₓ is the maximum pressure over the pressure cycle. Input of input fluid 346 into enclosed space 317 via lumen 345 and withdrawal of output fluid 348 from enclosed space 317 via lumen 347 may be sequential with one another, meaning the input of input fluid 346 into enclosed space 217 and withdrawal of output fluid 348 from enclosed space 317 does not occur simultaneously, in this implementation. In other implementations, two ports, such as ports 242, 244, may communicate with enclosed space 317, and fluid may be input through one or the two ports and withdrawn through the other of the two ports.

The pressure cycle of pressure p₀ within enclosed space 317 may be, for example, pressure cycle 500, 550, 600, 650, 700, 750, 800, 850, 900, 950 (see FIGS. 7A, 7B, 7C, 7D, 7E, 7F, 7G, 7H, 7I, 7J, respectively). Wound interface 315 may be formed of various plastics, and wound interface 315 may be deformation resistant to maintain generally its shape over the pressure range pₘᵢₙ≤p₀≤pₘₐₓ. A controller, such as controller 480 of wound therapy apparatus 400, may be in fluid communication with lumen 347 of port 342 to input the input fluid 346 into enclosed space 317 and to withdraw output fluid 348 from enclosed space 317 in order to vary the pressure p₀ within enclosed space 317, for example, according to pressure cycle 500, 550, 600, 650, 700, 750, 800, 850, 900, 950.

As illustrated in FIG. 5, wound therapy apparatus 300 includes layers 360, 370, and 380 within enclosed space 317. Various numbers of layers, such as layers 360, 370, 380, may be included in other implementations of wound therapy apparatus 300, and the layers may be arranged in various ways. Portions of layer 380, as illustrated, are secured to distal side 322 of member 320, and portions of distal side 382 of layer 380 are biased against skin surface 311 and wound bed 313. Layer 370 is biased between layer 380 and layer 360 with distal side 372 of layer 370 biased against proximal side 384 of layer 380, and proximal side 374 of layer 370 biased against distal side 362 of layer 360. Layer 360 is biased between layer 370 and spacer 330 with proximal side 364 of layer 360 biased against distal side 332 of spacer 330.

Proximal side 334 of spacer 330 is secured to distal side 322 of member 320 within enclosed space 317, as illustrated. Spacer 330 defines void 337 within spacer 330, and spacer 330 maintains layers 360, 370, 380 in biased engagement with one another, as illustrated. Spacer 330 may generally be a bilayer polymer bag with or without additional distribution channels that may be created by localized welding. Spacer 330 may optionally be welded at multiple points in the bilayer to limit distension of the void 337 when under pressure. The purpose of spacer 330, in this implementation, is to disperse input fluid 346 across the entire wound surface and to allow the withdrawal of output fluid 348 from throughout enclosed space 317. Wound interface 315 may have a variety of shapes and sizes ranging from circular, rectangular, ovoid, etc., and layers 360, 370, 380 and spacer 330 may conform in shape generally with the shape of wound interface 315.

Lumen 345 passes through port 342 and through proximal side 334 of spacer 330 into void 337, and input fluid 346 may be input via lumen 345 into void 337 or output fluid 448 may be withdrawn from void 337 through lumen 345.

For example, input fluid 346 may be input into void 337 through lumen 345, and input fluid 346 may then disperse within void 337 so that essentially the same pressure p₀ exists throughout void 337. Input fluid 346 may then flow from void 337 through spacer passages in distal side 332 of spacer 330, such as spacer passage 335, into layer 360. The spacer passages may be evenly distributed over distal side 332 of spacer 330 so that input fluid 346 is evenly distributed over proximal side 364 of layer 360 from void 337. Input fluid 346 may then flow through layer 360, through layer 370, and through layer passages, such as layer passage 385, in layer 380 to contact wound bed 313 as well as skin surface 311. The layer passages, which pass between proximal side 384 and distal side 382 of layer 380, may be evenly distributed over layer 380 so that input fluid 346 is evenly distributed over skin surface 311 and wound bed 313. Thus, for example, input fluid 346 may provide enhanced O₂ exposure to wound bed 313 and to skin surface 311. The pressure p₀ exists throughout enclosed space 317 including wound bed 313 and skin surface 311 because input fluid 346 and output fluid 348 may flow throughout enclosed space 317 including through spacer 330 and, thence, through layers 360, 370, 380.

Exudate 351 may flow from wound bed 313 through layer passages, such as layer passage 385, in layer 380 into layer 370, from layer 370 into layer 360, and from layer 360 through spacer passages, such as spacer passage 335, into void 337. Output fluid 348 including exudate 351 may flow from layers 380, 370 360 through spacer passages 335 into void 337, and output fluid 348 including exudate 351 may be withdrawn from void 337 through port 342 via lumen 345, in this implementation.

Layer 380 is formed of silicone, in this implementation, and wound bed 313 has the form of an incision with stitch 399. Silicone is known to have salutary effects on the healing of scarring from incisions. Of course, wound bed 313 may be any type of wound bed, and layer 380 may be formed of other materials, in various other implementations. Layer passages, such as layer passage 385, allow fluid exchange with wound bed 313 and skin surface 311 through layer 380, which may, for example, prevent maceration of skin 311. Silicone, as used herein, includes siloxane, various polysiloxanes, silicone-like materials, and various combinations thereof that may be generally solid. Silicone may have the chemical formula [R₂SiO]ₙ, where R is an organic group. Silicone may include, for example, silicone polymers having an average molecular weight in excess of 100,000 (e.g., between about 100,000 and about 10,000,000). Examples may include, but are not limited to, crosslinked siloxanes (e.g., crosslinked dimethicone or dimethicone derivatives), copolymers such as stearyl methyl-dimethyl siloxane copolymer, polysilicone-11 (a crosslinked silicone rubber formed by the reaction of vinyl terminated silicone and (methylhydro dimethyl)polysiloxane in the presence of cyclomethicone), cetearyl dimethicone/vinyl dimethicone crosspolymer (a copolymer of cetearyl dimethicone crosslinked with vinyl dimethyl polysiloxane), dimethicone/phenyl vinyl dimethicone crosspolymer (a copolymer of dimethylpolysiloxane crosslinked with phenyl vinyl dimethylsiloxane), and dimethicone/vinyl dimethicone crosspolymer (a copolymer of dimethylpolysiloxane crosslinked with vinyl dimethylsiloxane).

Layer 370 may include a layer of material that delivers therapeutics in a slow release manner, in this implementation. Such therapeutics may include, for example, silver ion based compounds or antibiotic for antimicrobial activity, local anesthetic for pain reduction, amniotic or placental derived cytokines and growth factors, hemostatics and coagulants to stop bleeding, oxygen generating and releasing compounds, exo- or endothermic reagents, etc.

Layer 360 may be made of a variety of materials including cotton gauze, polyester or polyamide fibers, or open-cell foams of polyurethane or polyvinyl alcohol. Layer 360 may optionally be augmented with a super absorbent polymer such as sodium polyacrylate, particularly when the intent is to lock the exudate within layer 360.

FIG. 6 illustrates exemplary wound therapy apparatus 400. As illustrated in FIG. 6, wound therapy apparatus 400 includes gas source 482 and liquid source 484 in fluid communication with controller 480, and controller 480 is in fluid communication with wound interface 415. Wound interface 415 is secured to skin surface 411 to define enclosed space 417 over a wound bed, such as wound bed 13, 113, 213, 313, as illustrated. Wound interface 415 may be formed, for example, similarly to wound interface 15, 115, 215, 315, and enclosed space 417 may be similar to enclosed space 17, 117, 217, 317, respectively.

Controller 480, in this implementation, includes control group 493 and canister 481, and control group 493 includes microcontroller 487 in operative communication with power source 498, user I/O 486, valve 488, pump 489, and pressure sensor 491 to control or monitor the operation of power source 498, valve 488, pump 489, pressure sensor 491, at least in part in response to the user inputs. Microcontroller 487 may include, for example, a microprocessor, memory, A/D converter, D/A converter, clock, I/O connectors, and so forth, and microcontroller may be configured for example, as a single chip or as an array of chips disposed about a circuit board, as would be readily recognized by those of ordinary skill in the art upon study of this disclosure.

Power source 498 may be, for example, mains electric or battery, and power source 498 may include, for example, a transformer, inverter, rectifier, or power filter. Valve 488 and pressure sensor 491 may be representative of a number of valves and a number of pressure sensors, respectively, in this illustration. Various communication pathways may be disposed about controller 480 to communicate electrical power from power source 498 to microcontroller 487, valve 488, pump 489, and pressure sensor 491 and to communicated data between microcontroller 487, valve 488, pump 489, and pressure sensor 491.

User I/O 486 may include various switches, push buttons, dials, and so forth, whether virtual or physical for obtaining user inputs that are then communicated to microcontroller 487 in order to allow the user to direct the operation of wound therapy apparatus 400. Various communication pathways such as electrical, electromagnetic (e.g. Bluetooth), optical (e.g. LASER, IR), and networked communications may be employed for communication between microcontroller 487 and user I/O 486. Microcontroller 487 controls the operation of wound therapy apparatus 400 including controller 480 based, at least in part, upon user inputs communicated to microcontroller 487 from user I/O 486. Microcontroller 487 may communicate date to user I/O 486 indicative of the operation of wound therapy apparatus 400, and user I/O 486 may display this data to the user.

As illustrated in FIG. 6, gas source 482 fluidly communicates gas 425 with control group 493 of controller 480, and liquid source 484 fluidly communicates liquid 485 with control group 493 of controller 480. Control group 493 of controller 480 as controlled by microcontroller 487 is operable to select gas 483 from gas source 482, liquid 485 from liquid source 484, or combinations of gas 483 from gas source 482 and liquid 485 from liquid source 484 as input fluid 446 that is input into enclosed space 417. Control group 493 of controller 840 as controlled by microcontroller 487 is operable to control the flow of input fluid 446 from controller 480 to enclosed space 417 of wound interface 415, the flow of output fluid 448 from enclosed space 417 of wound interface 415 to controller 480, and the exhausting of at least portions of output fluid 448 into the atmosphere, in this implementation, using valve 488, pump 489, and pressure sensor 491. By controlling the flow of input fluid 446 into enclosed space 417 and the withdrawal of output fluid 448 from enclosed space 417, controller 480 may cycle the pressure p₀ within enclosed space 417, for example, according to pressure cycle 500, 550, 600, 650, 700, 750, 800, 850, 900, 950 (see FIGS. 7A, 7B, 7C, 7D, 7E, 7F, 7G, 7H, 7I, 7J, respectively). Controller 480 may, for example, deliver Therapy Regimen 1, 2, 3 or 4, to the wound bed enclosed by wound interface 415 (see Example I).

Valve 488 may include a number of valves disposed about controller 480 and operable, for example, to select input fluid 446 as either gas 483 from gas source 482 or liquid 485 from liquid source 484, to control the flow of input fluid 446 from controller 480 to enclosed space 417 of wound interface 415, and to control the flow of output fluid 448 from enclosed space 417 of wound interface 415 to controller 480. Pressure sensor 491 may include a number of pressure sensors operable, for example, to monitor pressure at various locations in gas 483, liquid 485, input fluid 446, output fluid 448, or enclosed space 417 of wound interface 415. Microcontroller 487 may alter the operation of valve 488 in response to signals from pressure sensor 491. Input fluid 446 may be communicated under pressure at gas source 482 or liquid source 484, and pump 489 may be used to convey output fluid 448 from enclosed space 417 through canister 481.

Wound therapy apparatus 400 may include various fluid conveyances, for example hoses, pipes, valves, tubing, connectors, pressure regulators, and various other fittings, to communicate gas 483 and liquid 485 from gas source 482 and liquid source 484, respectively, to controller 480 and to communicate input fluid 446 and output fluid 448 between enclosed space 417 of wound interface 415 and controller 480.

Output fluid 448 passes through canister 481 as output fluid 448 is returned to controller 480 from wound interface 415 to capture exudate 419 or liquid, such as liquid 485, from output fluid 448 in chamber 499 of canister 481. Gaseous portions of output fluid 448 or gas displaced from chamber 499 of canister 481 by capture of liquid 485 or exudate 419 therein may then be discharged to the atmosphere from pump 489 of controller 480.

Exemplary pressure cycles 500, 550, 600, 650, 700, 750, 800, 850, 900, 950 are illustrated in FIGS. 7A, 7B, 7C, 7D, 7E, 7F, 7G, 7H, 7I, 7J, respectively, in which the pressure p₀ within an enclosed space, such as enclosed space 17, 117, 217, 317, 417 is plotted as a function of time t. In these exemplary pressure cycles, the pressure p₀ within the enclosed space is reduced by withdrawal of output fluid, such as fluid 48, 148, 248, 348, 448 from the enclosed space via a lumen, such as lumen 47, 145, 247, 345 and the pressure p₀ within the enclosed space is increased by input of fluid, such as input fluid 46, 146, 246, 346, 446 into the enclosed space via a lumen, such as lumen 45, 145, 245, 345. The fluid input into the enclosed space to increase the pressure p₀, such as input fluid 46, 146, 246, 346, 446 may include oxygen (O₂), and the O₂ concentration of the input fluid may be greater than that of atmospheric air. The wound bed, such as wound bed 13, 113, 213, 313, may thus be exposed to O₂ concentration greater than that of atmospheric air during at least portions of a number of pressure cycles in succession, in various implementations, which may increase the oxygen supply to the wound bed during therapy with resulting therapeutic benefits. The application of multiple pressure cycles to the wound bed with O₂ concentration greater than atmospheric air may increase the O₂ exposure of the wound bed and thus the time of oxygen therapy delivered to the wound bed. A controller, such as controller 480, may be used to cycle the pressure p₀ within the enclosed space, for example, according to pressure cycle 500, 550, 600, 650, 700, 750, 800, 850, 900, 950. Note that pressure cycles 500, 550, 600, 650, 700, 750, 800, 850, 900, 950 are exemplary only and not limiting, and one or more of these exemplary pressure cycles, various combinations of these exemplary pressure cycles or other pressure cycles may be delivered to the wound bed, in various implementations. The pressure cycle including the period, amplitude, and other characteristics of the pressure cycle may be altered over a sequence of pressure cycles.

As illustrated in FIG. 7A, pressure cycle 500 is initiated at time t₀ and pressure p₀=pₘₐₓ. According to exemplary pressure cycle 500, pressure p₀ reduces linearly at rate S₁ from time t₀ to time t₁, and then pressure p₀ reduces linearly at rate S₂ between time t₁ and time t₂ reaching pₘᵢₙ at time t₂. Pressure p₀ is then maintained at pₘᵢₙ between time t₂ and time t₃. Then pressure p₀ increases linearly at rate S₃ from pₘᵢₙ to pₘₐₓ between time t₃ and time t₄, as illustrated. Fluid input into the enclosed space between time t₃ and time t₄ to increase the pressure p₀ from pₘᵢₙ to pₘₐₓ may have an O₂ concentration greater than that of atmospheric air. Accordingly, the wound bed may be exposed to O₂ at a concentration greater than that found in atmospheric air during successive pressure cycles 500 when the pressure in each such pressure cycle is increased by fluid having an O₂ concentration greater than that of atmospheric air. Thus, the wound bed, in this example, may be exposed to O₂ concentration greater than that of atmospheric air at pressure pₘₐₓ between time t₃ and time t₄.

The pressure may be reduced between times t₀ and t₂ by withdrawal of output fluid from the enclosed space without any concurrent input of input fluid into the enclosed space. Similarly, the pressure may be increased between times t₃ and t₄ by input of input fluid into the enclosed space without any concurrent withdrawal of output fluid from the enclosed space. Finally, there is no input of input fluid into the enclosed space and concurrent withdrawal of output fluid from the enclosed space between times t₂ and t₃, in various implementations of pressure cycle 500. Essentially no fluid is input into the enclosed space and no fluid other than exudate is withdrawn from the enclosed space between time t₂ and time t₃, in various implementations of pressure cycle 500. A controller, such as controller 480 of wound therapy apparatus 400, may control the withdrawal of output fluid between times t₀ and t₂ and the input of input fluid between times t₃ and t₄.

In pressure cycle 500, for example, pₘₐₓ≈p_{amb} and pₘᵢₙ=p_{amb}−85 mm Hg. The time period t₂−t₀ may be approximately 40 s, and pressure p₀ is then held at pₘᵢₙ for t₃−t₂=240 s, followed by time period t₄−t₃=80 s, so that the period of pressure cycle 500 is t₄−t₀=360 s (6 minutes or 10 pressure cycles per hour). Various other implementations may deliver, for example 12 pressure cycles per hour, 4 pressure cycles per hour, or 3 pressure cycles per hour, according to exemplary pressure cycle 500. Slopes S₁ and S₂ may be selected to avoid creating pain and S₂ may be less than S₁, as rapid decreases below pₘₐₓ in pressure p₀ may be painful. For example, decreasing the pressure p₀ from p_{amb} to p_{amb}−40 mm Hg over time period t₁−t₀=10 s may be generally pain free followed by decreasing the pressure p₀ from p_{amb}−40 mm Hg to p_{amb}−85 mm Hg over t₂−t₁=30 s again to attempt to minimize pain.

In various other implementations, pressure p₀ may change at a single constant rate between time t₀ and time t₂ (i.e., S₁=S₂) or pressure p₀ may change at three or more rates between time t₀ and time t₂. Pressure cycle 500 may repeat starting at time t₄ (i.e., time t₄ is set to time t₀), or some other pressure cycle, such as pressure cycle 550, 600, 650, 700, 750, 800, 850, 900, 950 may then be initiated starting at time t₄. Pressure cycle 500 may remain essentially unchanged over successive cycles, or various parameters of pressure cycle 500, such as pₘₐₓ, pₘᵢₙ, S₁, S₂, t₃−t₂, t₄−t₀, may be altered over successive cycles.

An exemplary pressure cycle 550 is illustrated in FIG. 7B. As illustrated in FIG. 7B, pressure cycle 550 is initiated at time t₁₀ and pressure p₀≈pₘᵢₙ. Pressure p₀ increases linearly at rate S₁₁ from time t₁₀ to time t₁₁ reaching pₘₐₓ at time t₁₁. Pressure p₀ is then maintained at pₘₐₓ between time t₁₁ and time t₁₂, and then pressure p₀ decreases linearly at rate S₁₂ from pₘₐₓ to pₘᵢₙ between time t₁₂ and time t₁₃, as illustrated.

Input fluid that is input into the enclosed space between time t₁₀ and time tᵢᵢ in order to increase the pressure p₀ from pₘᵢₙ to pₘₐₓ may have an O₂ concentration greater than that of atmospheric air. Accordingly, the wound bed may be exposed to enhanced oxygen at pressure p₀ greater than pₘᵢₙ for time period t₁₃−t₁₀ in exemplary pressure cycle 550. Because generally p_{amb}≤pₘₐₓ the wound bed may be exposed to enhanced oxygen at pressure p₀ generally greater than or equal to ambient pressure p_{amb} for time period t₁₂−tᵢᵢ in exemplary pressure cycle 550.

The pressure p₀ may be increased between times t₁₀ and t₁₁ by input of input fluid into the enclosed space without any concurrent withdrawal of output fluid from the enclosed space. Similarly, the pressure p₀ may be decreased between times t₁₂ and t₁₃ by withdrawal of output fluid from the enclosed space without any concurrent input of input fluid into the enclosed space. Finally, there is no input of input fluid into the enclosed space and concurrent withdrawal of output fluid from the enclosed space between times tᵢᵢ and t₁₂, in various implementations of pressure cycle 550. A controller, such as controller 480 of wound therapy apparatus 400, may control the input of input fluid between times t₁₀ and t₁₁ and the withdrawal of output fluid between times t₁₂ and t₁₃.

In pressure cycle 550, for example, pₘₐₓ=p_{amb}+40 mm Hg and pₘᵢₙ=p_{amb}, approximately. The time period t₁₁−t₁₀ may be approximately 40 s, and pressure p₀ is then held at pₘₐₓ for approximately t₁₂−tᵢᵢ=240 s, followed by time period t₁₃ t₁₂=80 s approximately, so that the period of exemplary pressure cycle 550 is t₁₃ t₁₀=360 s (6 minutes or 10 pressure cycles per hour), approximately.

The pressure pₘₐₓ of pressure cycle 550 may be limited, for example in certain implementations, by the ability of the adhesive, such as adhesive 190, to secure wound interface to a skin surface, such as skin surface 11, 111, 211, 311. under pressure pₘₐₓ, which forces the wound interface away from the skin surface.

Pressure cycle 550 may repeat starting at time t₁₃ (i.e. time t₁₃ is set to time t₁₀), or some other pressure cycle, such as pressure cycle 500, 600, 650, 700, 750, 800, 850, 900, 950 may then be initiated starting at time t₁₃. Pressure cycle 550 may remain essentially unchanged over successive cycles, or various parameters of pressure cycle 550, such as pₘₐₓ, pₘᵢₙ, S₁₁, S₁₂, t₁₁−t₁₀, t₁₂−t₁₁, t₁₃−t₁₂, may be altered over successive cycles.

For example, in certain implementations, the controller, such as controller 480 of wound therapy apparatus 400, may deliver several pressure cycles according to pressure cycle 550 and then a pressure cycle according to pressure cycle 500 so that the pressure p₀ varies between pressures greater than ambient pressure p_{amb} that deliver enhanced oxygen (hyperbaric) to the wound bed and pressures less than ambient pressure p_{amb} that may remove exudate, such as exudate 51, 151, 251, 351, 419, from the wound bed or reseal the adhesive, such as adhesive 190, 390, to the skin surface. For example, pressure cycles 500, 550 may be combined so that time period t₁₃−t₁₀ is about 4 minutes and time period t₄- t₀ is about 2 minutes to deliver hyperbaric therapy to the wound bed for about ⅔ of the pressure cycle period of 6 minutes and to deliver suction therapy for about ⅓ of the pressure cycle period. When pressure cycles 500, 550 are so combined, the resultant pressure cycle is asymmetric with more time period spent delivering hyperbaric therapy and less time period spent delivering suction therapy, in this example.

Another exemplary pressure cycle 600 is illustrated in FIG. 7C. In exemplary pressure cycle 600, pressure p₀ decreases and increases continuously in a sinusoidal manner, as illustrated in FIG. 7C. As illustrated in FIG. 7C, pressure cycle 600 is initiated at time t₂₀ and pressure p₀≈pₘₐₓ. In this implementation, pressure p₀ decreases from time t₂₀ to time t₂₁ reaching pₘᵢₙ at time t₂₁, pressure p₀ then increases from pₘᵢₙ to pₘₐₓ between time t₂₁ and time t₂₂, and then pressure p₀ decreases from pₘₐₓ to pₘᵢₙ between time t₂₂ and time t₂₃. Pressure cycle 600 may repeat any number of times. Pressure cycle 600 may remain essentially unchanged over successive cycles, or various parameters of pressure cycle 600, such as pₘₐₓ, pₘᵢₙ, or the period t₂₂−t₂₀ of the pressure cycle may be altered over successive cycles. In other implementations, the pressure p₀ may decrease from pₘₐₓ to pₘᵢₙ in a sinusoidal (non-linear) manner, then maintained at a constant pressure pₘᵢₙ for some time period, and finally increase from pₘᵢₙ to pₘₐₓ in a sinusoidal manner.

Another exemplary pressure cycle 650 is illustrated in FIG. 7D. In exemplary pressure cycle 650, pressure p₀ decreases and then increases continuously as a triangular waveform, as illustrated in FIG. 7D. As illustrated in FIG. 7D, pressure cycle 650 is initiated at time t₃₀ and pressure p₀=pₘₐₓ. In this implementation, pressure p₀ decreases linearly from time t₃₀ to time t₃₁ reaching pₘᵢₙ at time t₃₁, and then pressure p₀ increases linearly from pₘᵢₙ to pₘₐₓ between time t₃₁ and time t₃₂ thus completing one pressure cycle. The next pressure cycle starts with pressure p₀ decreasing linearly from time t₃₂ to time t₃₃ reaching pₘᵢₙ at time t₃₃. Pressure cycle 650 may repeat any number of times. The fluid in the form of gas input to increase the pressure p₀ to pₘₐₓ may include O₂ at a concentration greater than that found in atmospheric air, and such increased O₂ may be delivered several times in succession by successive waveforms.

Another exemplary pressure cycle 700 is illustrated in FIG. 7E. As illustrated in FIG. 7E, pressure p₀ is altered stepwise (pulsatile) between pₘᵢₙ to pₘₐₓ over steps having period t₄₁−t₄₀, t₄₂−t₄₁, t₄₃−t₄₂, and t₄₄−t₄₃, as illustrated. The abrupt increase in pressure p₀ from pₘᵢₙ to pₘₐₓ in pressure cycle 700 may expel any residual exudate, such as exudate 51, 151, 251, 351, 419, out of lumen in communication with the enclosed space including tubing, such as tubing 49, in communication with the lumen in order to remove partial or full blockages caused by condensation or solidification of the exudate including other debris. In various implementations, pulses of fluid in the form of gas or liquid in general conformance to pressure cycle 700 may be introduced into the enclosed space to remove blockages from lumen, ports, or tubing in communication with the enclosed space. This may maintain the patency of the suction tubing and enable accurate sensing of pressure p₀ within the enclosed space by a sensor, such as sensor 491 of wound therapy apparatus 400. The magnitude of the step may be produced for example, by a high fluid flow rate or by a high-compliance reservoir balloon that is interposed between the fluid source and a solenoid valve that regulates input fluid delivered to the enclosed space, such as valve 488 of certain implementations of wound therapy apparatus 400. The pressure should be controlled to remain below a level that could breach the fluid-tightness of the wound interface. This is dependent on a number of factors including the characteristics of the adhesive that is used to anchor the protective covering. In various implementations, such blast relief pressure may be kept below about 30-40 mm Hg above ambient pressure pₐ.

Another exemplary pressure cycle 750 is illustrated in FIG. 7F. As illustrated in FIG. 7F, pressure p₀ decreases linearly from pₘₐₓ to pₘᵢₙ between times t₅₀ and t₅₁ and then to pressure p₀ increases exponentially (non-linearly) from pₘᵢₙ to pₘₐₓ between time t₅₁ and t₅₂. In exemplary pressure cycle 750, pressure p₀ is maintained constant at pₘₐₓ for time period t₅₃−t₅₂, for example, to deliver oxygen to the wound bed at pressure pₘₐₓ for at least time period t₅₃−t₅₂, as fluid with oxygen concentration greater than that of atmospheric air may be input between times t₅₂ and t₅₁ to increase the pressure p₀ from pₘᵢₙ to pₘₐₓ. The cycle 750 repeats starting at time t₅₃ with linear decrease in pressure p₀ from pₘₐₓ to pₘᵢₙ between times t₅₃ and t₅₄ followed by exponential increase from pₘᵢₙ to pₘₐₓ, as illustrated in FIG. 7F.

Another exemplary pressure cycle 800 is illustrated in FIG. 7G. As illustrated in FIG. 7G, pressure p₀ varies linearly from pₘᵢₙ to pₘₐₓ between time t₆₀ and t₆₁ and from pₘₐₓ to pₘᵢₙ between time t₆₂ and t₆₃. Fluid with oxygen concentration greater than that of atmospheric air may be input by the control group between times t₆₀ and t₆₁ to increase the actual pressure pₐ to maximum pressure pₘₐₓ. Pressure p₀ is maintained constant at pₘₐₓ for time period t₆₂−t₆₁, for example to deliver oxygen at pressure pₘₐₓ to the wound bed, and pressure p₀ is maintained constant at pₘᵢₙ for time period t₆₄−t₆₃, for example to withdraw exudate from the wound bed, in exemplary pressure cycle 800.

Another exemplary pressure cycle 850 is illustrated in FIG. 7H. As illustrated in FIG. 7H, pressure p₀ varies sinusoidally from pₘₐₓ to pₘᵢₙ between times t₇₀ and t₇₁ and from pₘᵢₙ to pₘₐₓ. between times t₇₂ and t₇₃. Pressure p₀ is maintained constant at pₘᵢₙ for time period t₇₂−t₇₁ and pressure p₀ is maintained constant at pₘₐₓ for time period t₇₄−t₇₃, in exemplary pressure cycle 850.

In exemplary pressure cycle 900, illustrated in FIG. 7I, pressure p₀ decreases linearly between times t₈₀ and t₈₁ increases linearly between times t₈₁ and t₈₂ and decreases linearly between times t₈₂ and t₈₃ and in a continuous sawtooth pattern. Note that maximum pressure pₘₐₓ is greater than ambient pressure p_{amb} in exemplary pressure cycle 900.

In exemplary pressure cycle 950, illustrated in FIG. 7J, pressure p₀ is initially at pₘ at time t₉₀. The pressure p₀ is increased from pₘᵢₙ to pₘₐₓ between times t₉₀ and t₉₁ by input of input fluid as liquid, such as liquid 485, into the enclosed space. The liquid which forms at least a portion of the input fluid, in this implementation, may provide various therapeutic benefits. The liquid may include, for example, saline solution, proteolytic enzyme solution, biofilm degradation solution, antibiotic lavage, amniotic fluid, platelet-enriched plasma, antibiotic, anesthetic, or other liquid having therapeutic benefits. In various implementations, 50 cc or more of liquid may be input into the enclosed space between times t₉₀ and t₉₁. The input fluid in the form of liquid remains within the enclosed space between times t₉₁ and t₉₂ to provide a therapeutic benefit to the wound bed, and the liquid is then withdrawn from the enclosed space including any pad, such as pad 150, or dressing, such as dressing 50, 250, within the enclosed space as the pressure p₀ is decreased from pₘₐₓ to pₘᵢₙ between times t₉₂ and t₉₃. The therapeutic benefit may include debridement, in various implementations. The decrease in pressure p₀ between times t₉₂ and t₉₃ may mark the beginning of a pressure cycle such as, for example, pressure cycle 500, 550, 600, 650, 700, 750, 850, 900. The decrease in pressure p₀ from pₘₐₓ to pₘᵢₙ between times t₉₂ and t₉₃ may remove 90% or more of the liquid from the enclosed space including any dressing, pad, or layers, such as layers 360, 370, 380, disposed therein, in certain implementations. Time period t₉₂−t₉₁ during which the liquid is within the enclosed space at pressure pₘₐₓ may range from about 2 minutes to about 1 hour, in various implementations. Time periods t₉₂−t₉₁ of less than 1 hour or time periods t₉₂−t₉₁ of only a few minutes may prevent maceration particularly when the skin surface is coated with adhesive such as cyanoacrylate. No input of input fluid into the enclosed space or withdrawal of output fluid from the enclosed space may occur between times t₉₁ and t₉₂, i.e, there is no flow through the enclosed space between times t₉₁ and t₉₂, in some implementations. In other implementations, liquid may pass through the enclosed space as input fluid and output fluid simultaneously i.e, the liquid is simultaneously input and withdrawn between times t₉₁ and t₉₂. Pressure cycle 950 may be intermittently interposed between other pressure cycles, such as pressure cycle 500, 550, 600, 650, 700, 750, 800, 850, 900. Pressure cycle 950 may be repeated several times in succession.

### Example I

Example I presents series of pressure cycles as used in exemplary wound therapy regimens and further demonstrates an exemplary application of these exemplary wound therapy regimens to wound therapy of a wound bed, such as wound bed 13, 113, 213, 313. The therapy regimens may be delivered to the wound bed using a wound therapy apparatus, such as wound therapy apparatus 10, 100, 200, 300, 400 that includes a wound interface, such as wound interface 15, 115, 215, 315, 415, that defines an enclosed space, such as enclosed space 17, 117, 217, 317, 417.

In this Example, dressing, such as dressing 50, 250, may be omitted from the wound bed during at least portions of the healing process. The absence of the dressing eliminates the need for dressing change and the associated pain and inhibition of the healing processes due to disruption of granulation tissue as well as the attendant costs for medical personnel and various consumables, and may allow for visual inspection of the wound bed and surrounding skin through transparent portions of the wound interface. Because no dressing is used in this implementation, the wound therapy apparatus may be employed until complete healing of the wound bed is achieved. The absence of the dressing, except, perhaps, in the initial exudative phase of wound bed, may permit, for example, lavage of wound bed as well as incubation of stem cells incubation of tissue stroma, proteolytic enzyme soaks, medical maggot debridement or a skin graft. The wound therapy apparatus may be employed until complete healing of the wound bed is achieved.

In Example I, N designates a pressure therapy according to exemplary pressure cycle 500 with O₂ input into the enclosed space between times t₃ and t₄ to increase the pressure within the enclosed space to pₘₐₓ. Note that humidity may be added to the O₂, or to other gas(es) in various pressure cycles to prevent drying of the wound bed. O designates a pressure therapy according to exemplary pressure cycle 550 with O₂ input into the enclosed space between t₁₀ and t₁₁ in order to increase the pressure within the enclosed space to pₘₐₓ with pₘₐₓ being greater than ambient pressure p_{amb} in pressure cycle 550 as used in Example I.

Therapy Regimens which are groups of four pressure cycles (four therapies) are as follows:

Therapy Regimen 1—N/N/N/N (four consecutive N therapies)
Therapy Regimen 2—N/N/N/O (three consecutive N therapies followed by one O therapy)
Therapy Regimen 3—N/O/N/O (N therapy alternating with O therapy)
Therapy Regimen 4 —N/O/O/O (one N therapies followed by three O therapies)

If each pressure cycle (either O therapy or N therapy) is delivered over 6 minutes, for example, each Therapy Regimen is then delivered over 24 minutes allowing the Therapy Regimen to be delivered 60 times a day. In general, at the early phase of wound treatment, relatively speaking, more N therapy may be used, as in exemplary Therapy Regimen 1 and exemplary Therapy Regimen 2, in order to remove exudate, such as exudate 51, 151, 251, 351, 419, and improve circulation. Once the exudative phase is over, the need for N therapy is diminished. At this point the therapy regimen may switch to N/O/N/O as in exemplary Therapy Regimen 3, and, lastly, O therapy would become the dominant therapy. An occasional N therapy may be interposed with a series of O therapies, as in exemplary Therapy Regimen 4, to reseat the wound interface onto the skin. An exemplary week of prescribed therapy Regimens may be:
Days 1-2: Therapy Regimen 1
Days 3-4: Therapy Regimen 2
Day 5-6: Therapy Regimen 3
Day 7: Therapy Regimen 4

Therapy Regimen 1, which is all N therapy, is used at the initiation of wound therapy, per Example I, as interstitial edema with large quantities of exudate may be present. The negative pressures p₀ of Therapy Regimen 1 may draw the exudate from the wound bed and may reduce the edema by withdrawing exudate from the wound bed that causes the edema. After two days of Therapy Regimen 1, the wound therapy changes from Therapy Regimen 1 to Therapy Regimen 2 that interposes O therapy with the N therapy. The use of O₂ under pressure p₀ generally greater than or equal to ambient pressure p_{amb} to deliver O₂ to the wound bed in the O therapy in the O therapy may aid in healing while the N therapy may continue to treat the edema by withdrawing exudate from the wound. Then, after two days of Therapy Regimen 2, the wound therapy changes from Therapy Regimen 2 to Therapy Regimen 3 that alternates O therapy with the N therapy as the wound continues to heal. The use of O₂ in the O therapy may aid in healing while the continued N therapy may continue to treat the edema by withdrawing exudate from the wound. Finally, at Day 7 per Example I, the wound therapy changes from Therapy Regimen 3 to Therapy Regimen 4, which is predominantly O therapy with one cycle of N therapy every four cycles. The negative pressures of the N therapy may re-adhere the wound interface to the skin thereby prolonging the life of the fluid-tight seal. Once the wound interface is unable to maintain a seal (typically due to skin shedding or adhesive failure), the wound interface may require replacement. Replacement is estimated to be once every 5 to 7 days depending on the location of the wound bed and individual variability. Note that a pressure cycle such as pressure cycle 950 may be included from time to time in any of Therapy Regimen 1, Therapy Regimen 2, Therapy Regimen 3, Therapy Regimen 4 to provide therapeutic liquid to the wound bed. The liquid may be, for example, saline solution, proteolytic enzyme solution, biofilm degradation solution, antibiotic lavage, amniotic fluid, platelet-enriched plasma, antibiotic, anesthetic, or other liquid having therapeutic benefits.

Thus, the progression, in Example I, is from initial use of N therapy that treats edema, to a mix of N therapy with O therapy that both treats edema and promotes healing, and, finally, to predominantly O therapy that promotes healing as the wound bed heals and the edema subsides. For example, Therapy Regimen 4 may be used when the wound is at least halfway healed and there is no longer any significant exudate.

It is assumed in Example 1 for explanatory purposes that the wound bed heals progressively between Day 1 and Day 7. Of course, healing may require other than a week, and, accordingly, the various Therapy Regimens, such as Therapy Regimens 1, 2, 3, and 4, may be continued for various lengths of time and may be combined as appropriate depending upon the condition of the wound bed. Therapy Regimens 1, 2, 3, and 4, may be linked with one another or with other Therapy Regimens in various ways, in various implementations. In other implementations, the Therapy Regimens, such as Therapy Regimens 1, 2, 3, 4, may have other patterns of pressure cycles, for example, O/O/O/O/. The Therapy Regimens, in other implementations, may have various numbers and types of cycles, such as pressure cycle 500, 550, 600, 650, 700, 750, 800, 850, 900, 950.

Accordingly, methods of use of the wound therapy apparatus, such as wound therapy apparatus 10, 100, 200, 300, 400, may include the step of securing sealingly a wound interface, such as wound interface 15, 115, 215, 315, 415, to the skin surface, such as skin surface 11, 111, 211, 311, 411, around a wound bed, such as wound bed 13, 113, 213, 313, forming an enclosed space, such as enclosed space 17, 117, 217, 317, 417, that is fluid-tight and enclosing the wound bed at the skin surface. Various adhesive(s), such as adhesive 90, 190, 290, 390, may be applied to the skin surface around the wound bed to protect the skin surface or to secure sealingly the wound interface to the skin surface. Once secured to the skin surface, the wound interface forms a fluid-tight enclosed space that encloses the wound bed perimeter of the wound bed at the skin surface. The methods of use may include delivering one or more pressure cycles to the wound bed, for example, pressure cycle 500, 550, 600, 650, 700, 750, 800, 850, 900, 950 illustrated in FIGS. 7A, 7B, 7C, 7D, 7E, 7F, 7G, 7H, 7I, 7J, respectively, and a number of pressure cycles may be grouped into a therapy regimen.

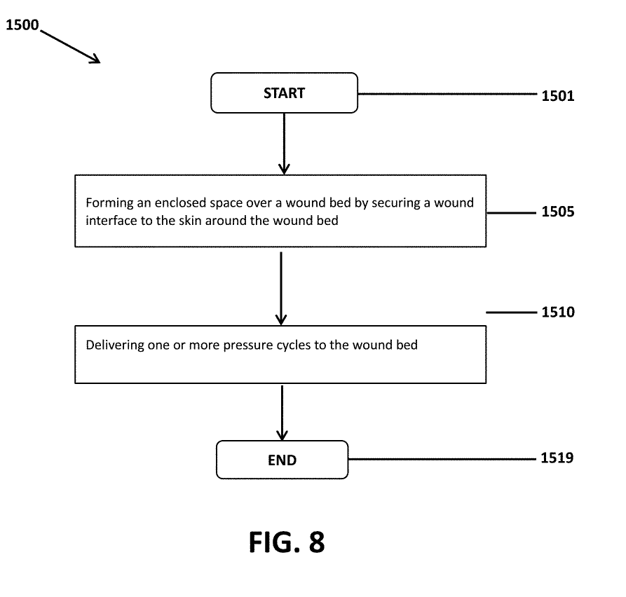

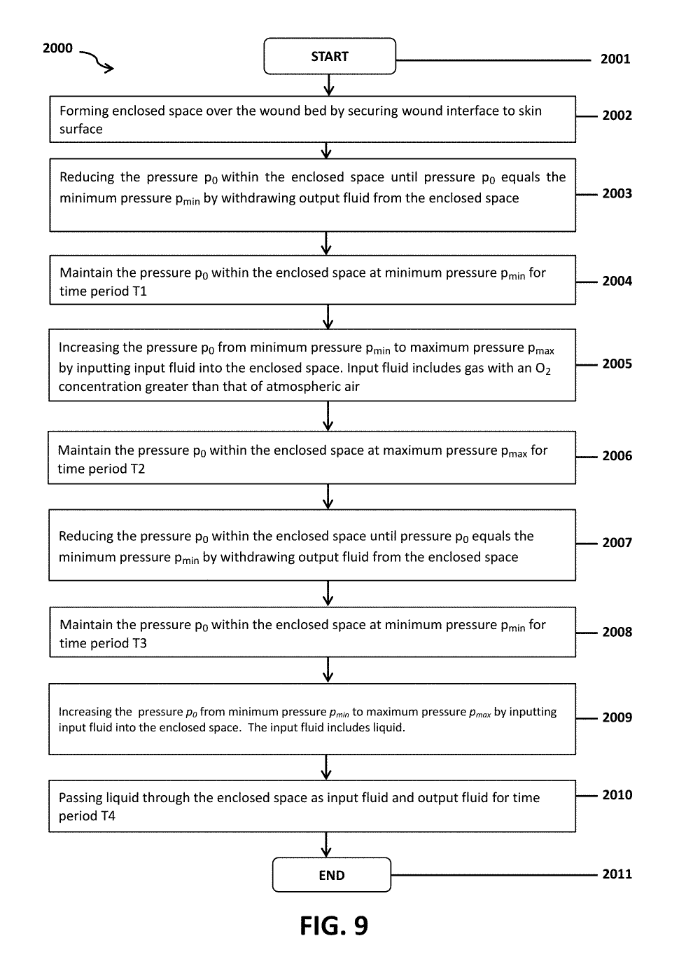

FIG. 8 illustrates an exemplary method of use of the wound therapy apparatus. Method 1500 is entered at step 1501. Step 1505 includes forming an enclosed space over a wound bed by securing a wound interface to the skin around the wound bed. At step 1510, one or more pressure cycles are delivered within the enclosed space to the wound bed. Method 1500 terminates at step 1519.

The wound interface, in certain implementations, may be sufficiently deformation resistant to accommodate distention of at least a portion of the wound bed into the enclosed space when the pressure p₀ within the enclosed space is less than ambient pressure p_{amb}. The methods of use may include the step of absorbing exudate, such as exudate 51, 151, 251, 351, 419 from the wound bed using a pad, such as pad 150, disposed about the enclosed space, and the step of evacuating exudate from the pad via a port, such as port 44, 142, 242, 244, 342, disposed about the wound interface. The pad may be in intermittent contact with the wound bed to intermittently absorb exudate from the wound bed, and the intermittent contact between the pad and the wound bed may result from periodic distention of the wound bed. The methods of use may include the step of varying periodically the pressure p₀ generally within the pressure range pₘᵢₙ≤p₀≤p_{amb}. The methods of use may include the step of varying periodically the pressure p₀ generally within the pressure range pₘᵢₙ≤p₀≤pₘₐₓ where p_{amb}<pₘₐₓ.

Various gaseous fluids, such as gas 483, may be introduced into the enclosed space or evacuated from the enclosed space as the pressure p₀ within the enclosed space is cycled. In various implementations, the fluid in the form of gas input to increase the pressure p₀ to pₘₐₓ may include O₂ at a concentration greater than that found in atmospheric air. In various implementations, the fluid in the form of gas used to increase the pressure p₀ to pₘₐₓ may include humidity to prevent drying of the wound bed. The composition of the gas(es) within the enclosed space may be controlled by input of gas(es) into the enclosed space, evacuation of gas(es) from the enclosed space, or both input of gas(es) into the enclosed space and withdrawal of gas(es) from the enclosed space, and the methods may thus include controlling the composition of the gas(es) within the enclosed space.

Various liquids, such as liquid 485, may be introduced into the enclosed space and subsequently evacuated from the enclosed space to provide a therapeutic benefit to the wound bed or to skin surrounding the wound bed.

The methods of use may include periodically varying the pressure p₀ and corresponding distention of the wound bed into the enclosed space from a relaxed state, such as relaxed state 193, into a distended state, such as distended state 194, and release of the wound bed from the distended state to the relaxed state thereby massaging the wound bed.

The methods of use may include the step of distending the wound bed into communication with the pad or the step of releasing the wound bed from communication with the pad. Distending the wound bed into communication with the pad may communicate exudate from the wound bed into the pad, and removing the wound bed from contact with the pad may prevent integration of the pad with the wound bed. Exudate may be withdrawn from the enclosed space via the port that communicates fluidly with the pad. The methods of use may include observing the wound bed within the enclosed space through portions of the wound interface formed of transparent material.

The methods of use may include omitting a dressing, such as dressing 50, 250, from the wound bed. The methods of use may include providing the dressing within the wound bed at early stages of treatment of the wound bed. The methods of use may include distributing pressure p₀ within the enclosed space evenly over the wound bed thereby preventing the creating of a pressure about the wound bed resulting in the decreasing of blood flow proximate the wound boundary.

Another exemplary method of use of the wound therapy apparatus is illustrated by process flow chart in FIG. 14. Operational method 2000 as illustrated in FIG. 14 and the associated description is exemplary only. As illustrated in FIG. 14, operational method 2000 is entered at step 2001. At step 2002, the wound interface of the wound therapy apparatus is secured to the skin surface forming the enclosed space over the wound bed. At step 2003, output fluid is withdrawn from the enclosed space thereby reducing the pressure p₀ within the enclosed space until p₀ equals the minimum pressure pₘᵢₙ. Pressure p₀ within the enclosed space may then be maintained at minimum pressure pₘᵢₙ for time period T₁, as per step 2004. For example, time period T₁ may be about 3 to 5 minutes. At step 2005, input fluid is input into the enclosed space thereby increasing the pressure p₀ from minimum pressure pₘᵢₙ, to maximum pressure pₘₐₓ. The input fluid input into the enclosed space at exemplary step 2005 to increase the pressure p₀ from minimum pressure pₘᵢₙ to maximum pressure pₘₐₓ comprises a gas with an O₂ concentration greater than that of atmospheric air.

At step 2006, the maximum pressure pₘₐₓ may be about ambient pressure p_{amb}, maximum pressure pₘₐₓ may be greater than ambient pressure p_{amb}, or maximum pressure pₘₐₓ may be less than ambient pressure p_{amb}, in various implementations. Pressure p₀ within the enclosed space may then be maintained at maximum pressure pₘₐₓ for time period T₂, as per exemplary step 2006. For example, time period T₂ may be about 1-3 minutes.

As illustrated in FIG. 14, output fluid is withdrawn from the enclosed space at step 2007 to reduce the pressure p₀ within the enclosed space until p₀ equals the minimum pressure pₘᵢₙ. Pressure p₀ within the enclosed space may then be maintained at minimum pressure pₘᵢₙ for time period T₃, as per step 2008. Because the fluid input into the enclosed space at step 2005 comprises a gas with an O₂ concentration greater than that of atmospheric air, the wound bed is exposed to gas with an O₂ concentration greater than that of atmospheric air throughout steps 2006, 2007, and 2008, in exemplary operational method 2000.

At step 2009, input fluid is input into the enclosed space to increase the pressure p₀ from minimum pressure pₘᵢₙ to maximum pressure pₘₐₓ. The input fluid at step 2009 comprises a liquid, in exemplary operational method 2000.

Output fluid is withdrawn from the enclosed space and input fluid is input into the enclosed space sequentially in performing steps 2003, 2004, 2005, 2006, 2007, 2008 and 2009, in exemplary operational method 2000, so that either input fluid is being input or output fluid is being withdrawn. Input fluid is not input at the same time output fluid is being withdrawn in performing steps 2003, 2004, 2005, 2006, 2007, 2008 and 2009 of exemplary operational method 2000.

At step 2010, liquid is then passed through the enclosed space for time period T₄. The liquid may be sequentially input into the enclosed space and then withdrawn from the enclosed space or the liquid may be simultaneously input into the enclosed space and withdrawn from the enclosed space, at step 2010. Liquid may be input in pulses to purge blockages within various passages that fluidly communicate with the enclosed space, at step 2010. At step 2010, the liquid may flush out the enclosed space including the wound bed and dressing, remove bioburden or exudate, cleanse the wound bed, hydrate the wound bed, for example. At step 2010, the liquid may be input and withdrawn by instillation (steady flow). Exemplary operational method 2000 then terminates at step 2011.

Liquid may be input into the enclosed space at step 2010 by being sucked in from a source, such as source 84, by pressure p₀ within the enclosed space less than ambient pressure p_{amb}. As liquid fills the enclosed space, the pressure p₀ may tend toward ambient pressure p_{amb} reaching ambient pressure p_{amb} when the enclosed space is filled by liquid. In certain implementations, there is no energy gradient between the liquid source and the enclosed space other than pressure difference p_{amb}−p₀ so that liquid flow into the enclosed space ceases once p₀=p_{amb} generally, thus preventing overfilling of the enclosed space that may dislodge the wound interface. In other implementations, the controller may limit the pressure p₀ of the liquid within the enclosed space for example to about ambient pressure p_{amb} in order to prevent dislodgement of the wound interface.

Exemplary method 2000 may be repeated any number of times with various combinations of steps 2003, 2004, 2005, 2006, 2007, 2008, 2009, 2010. Note that minimum pressure pₘᵢₙ and maximum pressure pₘₐₓ may change between steps 2003, 2004, 2005, 2006, 2007, 2008, 2009, 2010, and times T₁, T₂, T₃, T₄ as well as minimum pressure pₘᵢₙ and maximum pressure pₘₐₓ may be altered during various repetitions of method 2000.

Each pressure cycle may be augmented with an additional therapeutic benefit through the relief of the suction pressure pₘᵢₙ by gas having enhanced oxygen content or by liquid having therapeutic benefit. This may increase oxygen supply or add another therapy to the wound bed, and may have the effect of creating at least one additional new therapy of many hours daily without reducing the overall duration of the NWPT therapy.

The methods and apparatus disclosed herein, for example, allow for including other therapies in the around-the-clock therapy regimen to “concentrate” or “condense” the therapy day and directly accelerate healing, as if more therapy days had elapsed, and to do so without changing dressing. For example, the result of adding extra hours of therapy without reducing the pre-existing hours of pressure therapy, as if we have increased a 24-hour day for therapy to 32 hours or so.

There are only 24 hours in a day available for wound treatment and it is a fight against the clock to reestablish normal oxygenation and blood flow while gaining the upper hand on microbial overgrowth. If saline instillation were desired and saline instillation lasts 30 minutes, then the pressure therapy for that day is reduced to 23.5 hours. If it is desired to add proteolytic enzyme soaks of 2 hours, then the pressure therapy is further reduced to 21.5 hours, and so on. Since pressure therapy for chronic wound already lasts many weeks at substantial cost per week, the ability to add beneficial therapy without reducing the duration of pressure therapy, as disclosed herein, may be a therapeutic advance in the care of wound beds.

In various implementations, the methods of wound therapy and related apparatus may be used in humans, or, alternatively, for veterinary purposes. While the preceding discussion has focused on wound therapy, it should be recognized that the wound therapy methods and related apparatus and compositions of matter disclosed herein may have applications in other areas of human or veterinary medicine.

The foregoing discussion along with the Figures discloses and describes various exemplary implementations. These implementations are not meant to limit the scope of coverage, but, instead, to assist in understanding the context of the language used in this specification and in the claims. Upon study of this disclosure and the exemplary implementations herein, one of ordinary skill in the art may readily recognize that various changes, modifications and variations can be made thereto without departing from the spirit and scope of the inventions as defined in the following claims.

What is claimed is:

1. An apparatus for wound therapy, comprising:
a wound interface sealingly engaged with a skin surface to define an enclosed space surrounding a wound bed at the skin surface, the enclosed space is fluid-tight, the enclosed space evacuated to a pressure pₘᵢₙ less than ambient pressure p_{amb} and a condition of essentially no fluid passing through the enclosed space;
at least one port formed about the wound interface in fluid communication through the wound interface with the enclosed space; and
fluid input into the enclosed space via the at least one port to increase the pressure p₀ within the enclosed space from the minimum pressure pₘᵢₙ to a maximum pressure pₘₐₓ, the fluid comprising either a liquid or a gas having an O₂ concentration greater than atmospheric air.

2. The apparatus of claim 1, the gas consisting essentially of medical grade O₂.

3. The apparatus of claim 1, the gas consisting essentially of humidity combined with medical grade O₂.

4. The apparatus of claim 1, the wound interface being sufficiently deformation resistant to accommodate distention of the wound bed into the enclosed space at pressure p₀ less than ambient pressure p_{amb}.

5. The apparatus of claim 1, the wound interface comprising a sheet adhesively secured around the wound bed.

6. The apparatus of claim 1, further comprising:
a pad disposed within the enclosed space to contact the wound bed intermittently, the pad in fluid communication with the port to withdraw the exudate.

7. The apparatus of claim 1, further comprising:
a pulse of the fluid input into the enclosed space to expel debris from lumen in fluid communication with the enclosed space, the debris comprising exudate.

8. The apparatus of claim 1, wherein pₘₐₓ=p_{amb} approximately.

9. The apparatus of claim 1, wherein pₘᵢₙ=p_{amb}−175 mm Hg approximately.

10. An apparatus for wound therapy, comprising:
a wound interface sealingly engageable with the skin to define an enclosed space surrounding a wound bed that is fluid-tight; and
at least one port formed about the wound interface to communicate fluidly through the wound interface with the enclosed space to vary periodically a pressure p₀ within the enclosed space in a pressure cycle between a minimum pressure pₘᵢₙ and a maximum pressure pₘₐₓ where pₘᵢₙ≤p₀≤pₘₐₓ and where pₘᵢₙ≤p_{amb}≤pₘₐₓ by consecutive withdrawal of fluid from the enclosed space and input of fluid into the enclosed space through the at least one port, the fluid input into the enclosed space to increase the pressure p₀ within the enclosed space from the minimum pressure pₘᵢₙ to the maximum pressure pₘₐₓ has an O₂ concentration greater than atmospheric air.

11. The apparatus of claim 10, the wound interface being sufficiently deformation resistant to accommodate distention of the wound bed into the enclosed space at pressure p₀ less than ambient pressure p_{amb}.

12. The apparatus of claim 8, the wound interface comprising a sheet adhesively secureable to skin around the wound bed.

13. The apparatus of claim 10, further comprising:
a pad disposed within the enclosed space to contact the wound bed during a portion of the pressure cycle in order to absorb exudate from the wound bed, the pad in fluid communication with the port to withdraw the exudate from the pad through the port.

14. The apparatus of claim 10, wherein pₘₐₓ=p_{amb}+30 mm Hg approximately.

15. The apparatus of claim 10, wherein pₘₐₓ=p_{amb} approximately.

16. The apparatus of claim 10, further comprising:
the minimum pressure pₘᵢₙ is less than ambient pressure p_{amb} and the maximum pressure pₘₐₓ is greater than ambient pressure p_{amb}, the pressure p₀ is greater than ambient pressure p_{amb} for about ⅔ of a time period of the pressure cycle and the pressure p₀ is less than ambient pressure p_{amb} for about ⅓ of the time period of the pressure cycle.

17. The apparatus of claim 10, further comprising:
a therapy regimen, the therapy regimen comprising at least a sequence of pressure cycles of the pressure p₀ within the enclosed space, the sequence selected from:
Sequence 1—N/N/N/N
Sequence 2—N/N/N/O
Sequence 3—N/O/N/O
Sequence 4 —N/O/O/O
in pressure cycle O the pressure p₀ is varied between pₘᵢₙ and pₘₐₓ with pₘᵢₙ≈p_{amb} and p_{amb}<pₘₐₓ, and
in pressure cycle N the pressure p₀ is varied between pₘᵢₙ and pₘₐₓ with pₘᵢₙ<p_{amb} and pₘₐₓ≈p_{amb}.

18. The apparatus of claim 17, the therapy regimen comprising Sequence 1 followed by Sequence 2 followed by Sequence 3 followed by Sequence 4.

19. The apparatus of claim 17, further comprising:
the sequence of pressure cycles further comprising a liquid input into the enclosed space to increase the pressure p₀ within the enclosed space from the minimum pressure pₘᵢₙ to the maximum pressure pₘₐₓ.

20. The apparatus of claim 19, further comprising:
the liquid comprising one or more materials selected from the group consisting of saline solution, proteolytic enzyme solution, biofilm degradation solution, antibiotic lavage, amniotic fluid, platelet-enriched plasma, antibiotic solution, and anesthetic solution.

21. The apparatus of claim 19, the liquid is disposed within the enclosed space for a time period, there being no input of fluid into the enclosed space and there being no withdrawal of fluid from the enclosed space during the time period.

22. The apparatus of claim 21, the time period being between about 2 minutes and about 1 hour.

23. The apparatus of claim 10, the at least one port comprising:
a port through which fluid is input into the enclosed space; and
a second port through which fluid is withdrawn from the enclosed space.

24. The apparatus of claim 23, the fluid comprising:
liquid is input into the enclosed space through the port; and
liquid is withdrawn from the enclosed space through the second port.

25. A method of wound therapy, comprising the steps of:
forming an enclosed space surrounding a wound bed by engaging sealingly a wound interface to a skin surface, the enclosed space being fluid-tight;
evacuating the enclosed space to a pressure pₘᵢₙ less than ambient pressure p_{amb} and a condition of essentially no fluid passing through the enclosed space; and
increasing the pressure p₀ within the enclosed space from the minimum pressure pₘᵢₙ to a maximum pressure pₘₐₓ by inputting a gas having an O₂ concentration greater than that of atmospheric air into the enclosed space following the step of evacuating the enclosed space.
